(19) United States
(12) Patent Application Publication
(10) Pub. No.: US 2017/0079633 A1
(43) Pub. Date: Mar. 23, 2017

(54) DEVICE AND METHODS OF INVERTING A DIVERTICULUM

(71) Applicant: Empire Technology Development LLC, Wilmington, DE (US)

(72) Inventors: Todd Dickson, Santa Clara, CA (US); Abby George, Sunnyvale, CA (US); Worth Walters, Los Gatos, CA (US); Daniel Hawkins, Pleasanton, CA (US)

(21) Appl. No.: 14/858,979

(22) Filed: Sep. 18, 2015

(51) Int. Cl. A61B 17/00; A61B 17/12

(52) U.S. Cl. CPC A61B 17/00234; A61B 17/12; A61B 2017/00358; A61B 2017/00292; A61B 2017/00818

(57) ABSTRACT

Several embodiments disclosed herein relate to apparatus and methods for treating a diverticulum. In some examples, disclosed is a device for inverting a diverticulum including a device body, a pushing apparatus that includes a rod and a blunt end, and a user actuated structure that provides control of the pushing apparatus. Also disclosed is a method for inverting a diverticulum. The method can include positioning a device along an outer wall of a colon at a diverticulum, inverting the outer surface of the diverticulum with the distal end of the device, and then actuating the user actuated structure such that the pushing apparatus interacts with the diverticulum to further invert the diverticulum.

### BACKGROUND

An outpouching of the colon or other body lumen, called a diverticulum, can become the site for inflammation known as diverticulitis, microperforation and/or bleeding. Current treatments may involve the surgical removal of segments of the body lumen. For extreme cases of diverticulitis, treatment can involve colon resection and placement of a colostomy. This approach results in significant healthcare costs and substantial pain for patients.

### SUMMARY

Disclosed is a device for inverting a diverticulum. In some embodiments, the device includes a device body, a pushing apparatus including a rod and a blunt end, wherein the rod is disposed coaxially within the device body and the blunt end is located at the distal end of the rod, and a user actuated structure that provides control of the pushing apparatus.

In other embodiments, the device includes a pushing apparatus that is movable along the length and relative to the device body, and wherein the user actuated structure translates the pushing apparatus in a distal direction such that the rod extends from the distal end of the device body. In other embodiments, the pushing apparatus is spring loaded and the user actuated structure comprises a button or trigger configured to interact with the spring loaded pushing apparatus when actuated.

In other embodiments, the device includes a rod comprising a lumen and a blunt end comprising an expandable balloon, wherein the user actuated structure is a syringe configured to inject and withdraw fluid from the lumen to inflate or deflate the balloon. In other embodiments, the user actuated structure is a button or trigger activated syringe. In other embodiments the balloon is oval-shaped.

In other embodiments, the device includes a blunt end comprising a basket capable of expanding or collapsing as the pushing apparatus is extended or withdrawn from the distal end of the device body. In other embodiments, the basket is composed of a metal with elastic properties such as nitinol or cobalt-chromium. In other embodiments, the basket is composed of a polymer with elastic properties such as polyurethane, polyethylene terephthalate, or polyethyleneoxide. In other embodiments, the basket is oval-shaped when expanded. In other embodiments, the user actuated structure is a sliding member, button or a trigger.

Also disclosed is a method for inverting a diverticulum. In some embodiments, the method includes positioning a device along an outer wall of a colon at a diverticulum, wherein the device comprises a device body, a pushing apparatus, and a user actuated structure, wherein the device body is disposed about the pushing apparatus and the user actuated structure is configured to interact with the pushing apparatus. In some embodiments, the method includes inverting the outer surface of the diverticulum with the distal end of the device such that the distal end of the device is partially disposed within the inverted portion of the diverticulum. In some embodiments, the embodiment includes actuating the user actuated structure such that the pushing apparatus interacts with the diverticulum to further invert the diverticulum.

In other embodiments, the method includes a pushing apparatus comprising a rod and a blunt end attached to the distal end of the rod, wherein actuating the user actuated structure moves the pushing apparatus in a distal direction such that the blunt end extends from the distal end of the device body and further inverts the diverticulum.

In other embodiments, the method includes a pushing apparatus that is spring loaded and a user actuated structure that has a button or trigger configured to interact with the spring loaded pushing apparatus such that actuating the user actuated structure comprises depressing the button or activating the trigger.

In other embodiments, the method further includes retracting the pushing apparatus such that the rod and blunt end move in a proximal direction such that the rod withdraws into the device body.

In other embodiments, the method includes a pushing apparatus comprising a lumen coaxially disposed within the device body and an expandable balloon located at the distal end of the lumen. In other embodiments, the user actuated structure is a syringe configured to interact with the proximal end of the lumen and actuating the user actuated structure to further invert the diverticulum includes depressing a plunger of the syringe such that fluid is injected into the lumen and inflates the expandable balloon.

In other embodiments, the method further includes retracting the plunger of the syringe such that fluid is pulled out of the lumen and deflates the expandable balloon.

In other embodiments, the method includes an expandable balloon that is oval-shaped and the further includes the step of rotating the inflated expandable balloon such that the cross-section of the inverted diverticulum becomes increasingly circular.

In other embodiments, the method includes a pushing apparatus that includes a rod and a basket attached to the distal end of the rod, wherein actuating the user actuated structure to further invert the diverticulum includes moving the pushing apparatus in a distal direction such that the basket extends from the distal end of the device body and expands.

In other embodiments, the method includes retracting the pushing apparatus such that the rod and the basket move in a proximal direction such that the basket collapses into the distal end of the device body.

In other embodiments, the method includes a basket that is oval-shaped and the method further includes the step of rotating the expanded basket such that the cross-section of the inverted diverticulum becomes increasingly circular.

In other embodiments, the method includes a user actuated structure that is a sliding member, button, or a trigger.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A illustrates a side view of an embodiment of a device for inverting diverticulum with all of the components of the device exposed.

FIG. 1B illustrates a side view of the device illustrated in FIG. 1A with a few of the components retracted.

FIGS. 1C-K illustrate a plurality of views of a method for treating diverticulum disease using the device of FIG. 1A.

FIG. 1L illustrates a flowchart of an embodiment of the method for treating diverticulum disease illustrated in FIGS. 1C-K.

FIG. 2A illustrates a side view of another embodiment of a device for inverting diverticulum.

FIGS. 2B-H illustrate a plurality of views of a method for treating diverticulum disease using the device of FIG. 2A.

FIG. 3A illustrates a side view of another embodiment of a device for inverting diverticulum.

FIG. 3B illustrates a top view of an embodiment of a closure clip in a relaxed configuration wherein the closure clip can be used with the devices illustrated in FIGS. 1A, 2A, and 3A.

FIG. 3C illustrates a side view of the closure clip of FIG. 3B when it is loaded on any of the devices illustrated in FIGS. 1A, 2A, and 3A.

FIG. 3D illustrates a side view of the closure clip of FIG. 3B as it is delivered to a target site.

FIG. 3E illustrates a side view of an embodiment of a clip tube of the device of FIG. 3A.

FIG. 3F illustrates the clip tube of FIG. 3E that is loaded with the closure clip illustrated in FIG. 3D.

FIGS. 3G-I illustrate a plurality of views of the distal end of an embodiment of a push rod of the device of FIG. 3A.

FIG. 3J illustrates a side view of the clip tube of FIG. 3E and push rod of FIGS. 3G-I that is loaded with the closure clip illustrated in FIG. 3D.

FIG. 3K illustrates a side view of the apparatus of 3J where the closure clip is configured for delivery as illustrated in FIG. 3E.

FIG. 3L illustrates an embodiment of a sheath of the device of FIG. 3A.

FIGS. 3M-S illustrate a plurality of views of a method for treating diverticulum using the device of FIG. 3A.

FIGS. 4A-B are side views of dimensional presentations of diverticula and their inverted counterparts.

FIG. 5 is a side view of an existing device for diverticulum inversion used in a method for treating diverticulum disease.

FIGS. 6A-C are side views of a device for diverticulum inversion with an extendable atraumatic tip used in a method for treating diverticulum disease.

FIGS. 7A-B are side views of a device for diverticulum inversion with inflatable balloon tip used in a method for treating diverticulum disease.

FIGS. 8A-B are side view of a device for diverticulum inversion with expandable tip used in a method for treating diverticulum disease.

FIG. 9 is a side view of a device for diverticulum inversion with a spring-activated extendable atraumatic tip used in a method for treating diverticulum disease.

FIGS. 10A-B are top and side views of an existing device for diverticulum inversion used on an inverted diverticulum where the ostium is oval in shape.

FIGS. 11A-F are side and top views of a device for diverticulum inversion with an ellipsoid expandable tip used in a method for treating diverticulum disease.

FIGS. 12A-D are side, end, and top views of a device for diverticulum inversion with an ellipsoid inflatable tip used in a method for treating diverticulum disease.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Disclosed herein are methods and devices for treating diverticular disease. The method can include positioning a device along an outer wall of a colon at a diverticulum, inverting the outer surface of the diverticulum, and actuating the user actuated structure such that the pushing apparatus interacts with the diverticulum to further invert the diverticulum. The device can include a device body, a pushing apparatus including a rod and a blunt end, wherein the rod is disposed coaxially within the device body and the blunt end is located at the distal end of the rod, and a user actuated structure that provides control of the pushing apparatus.

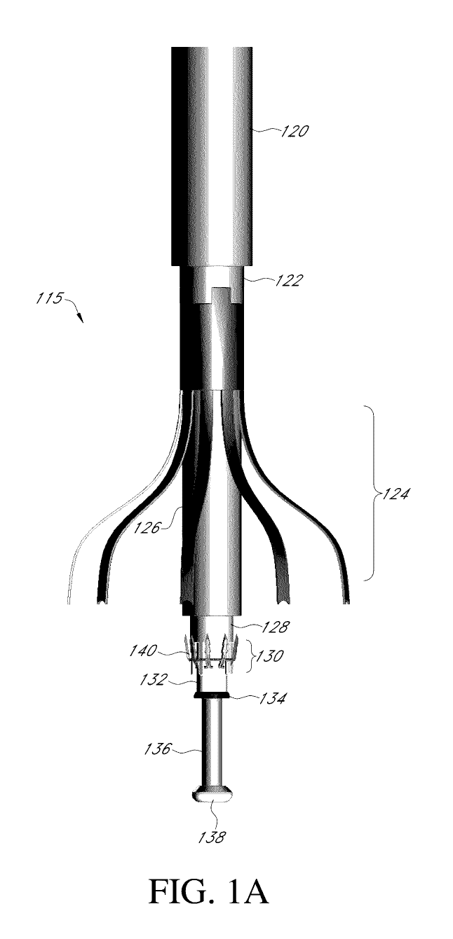

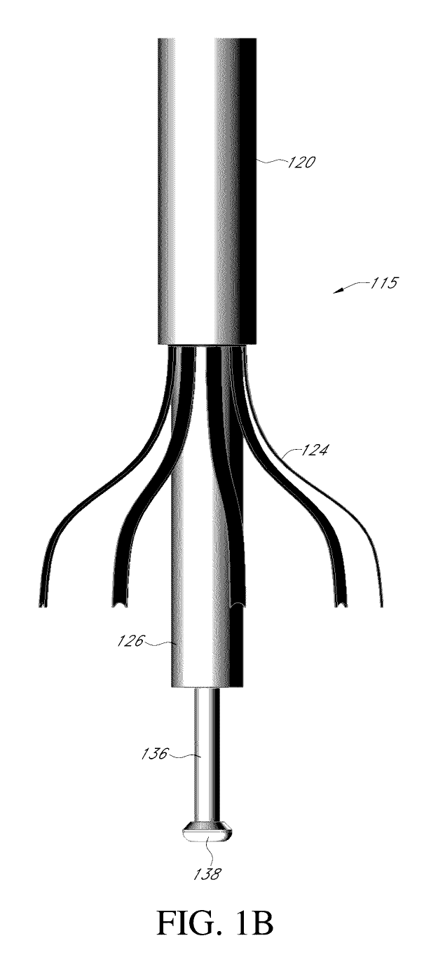

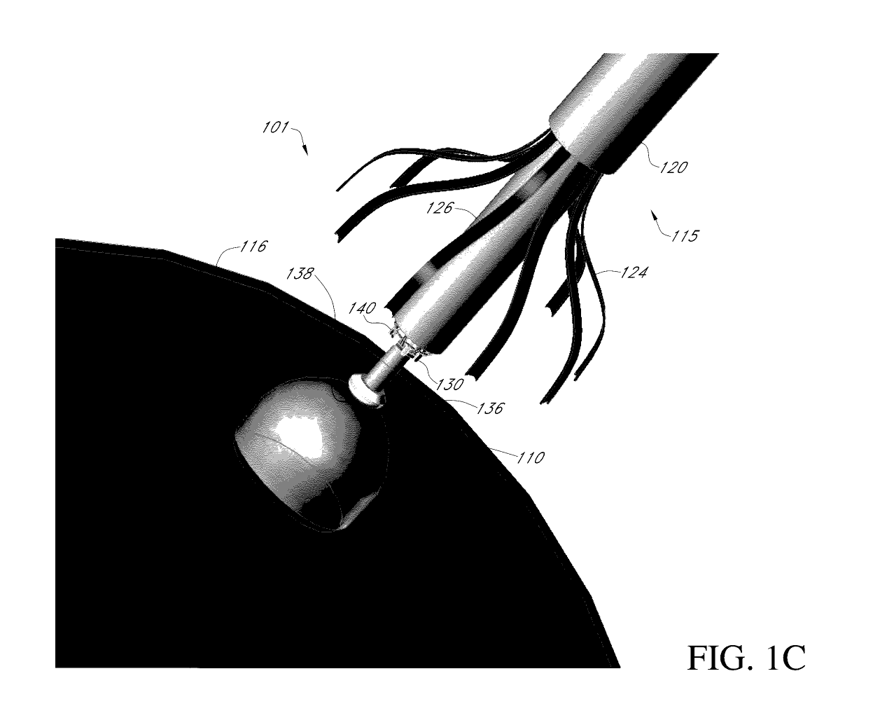

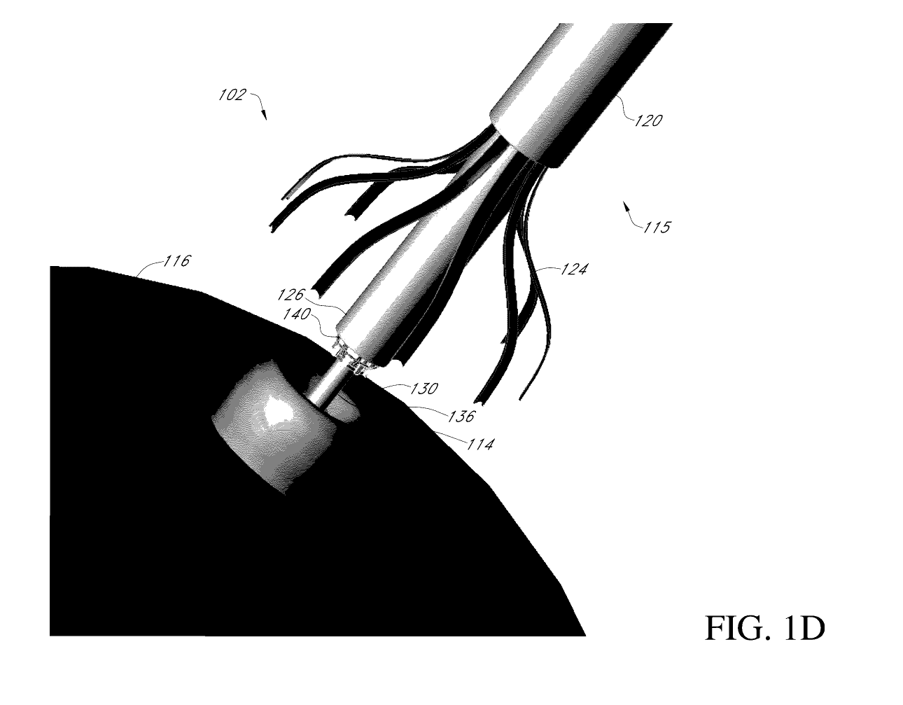

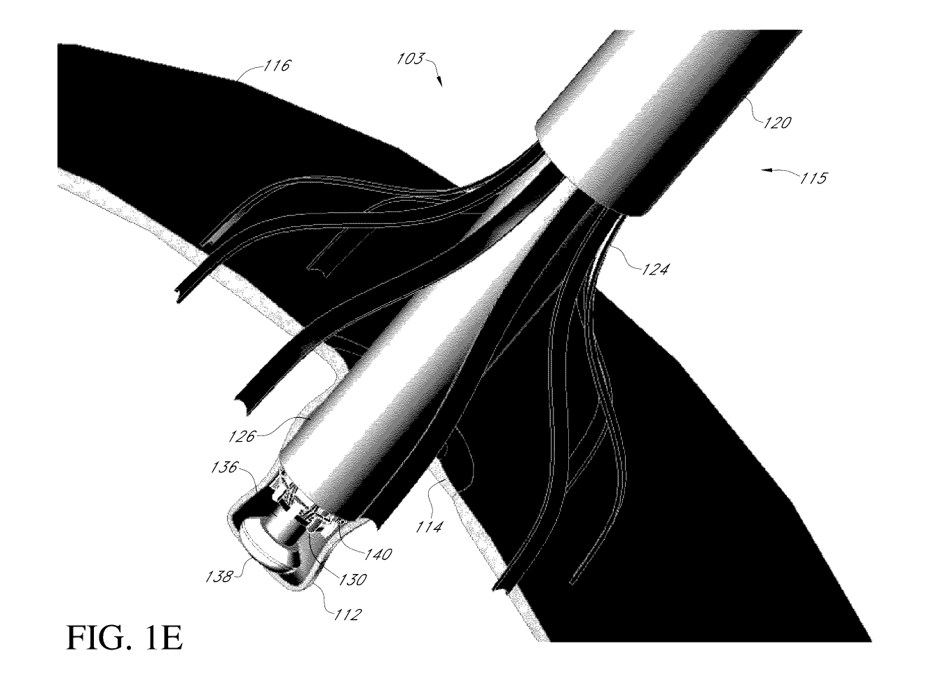

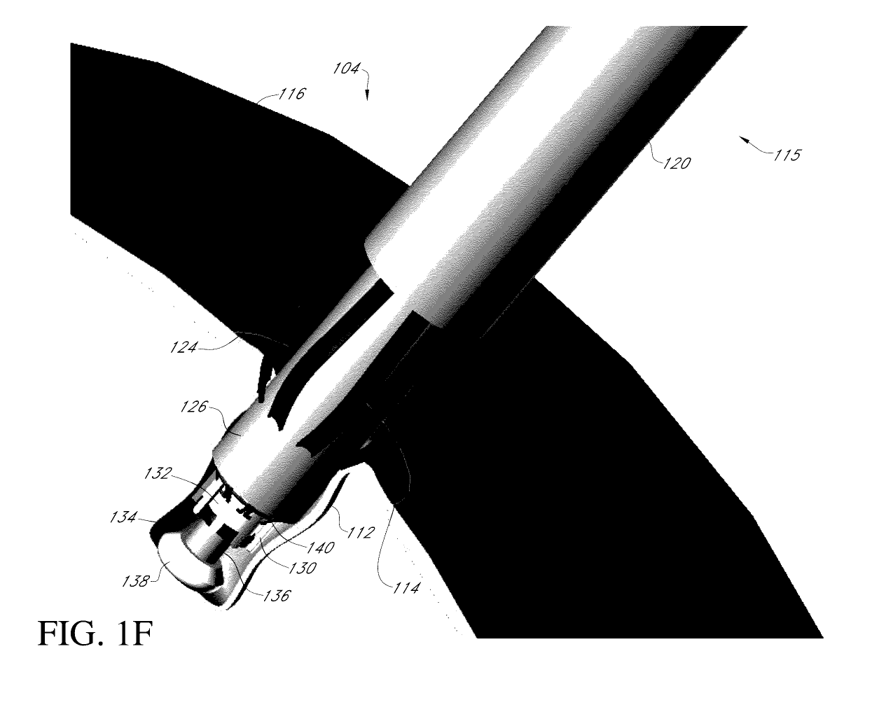

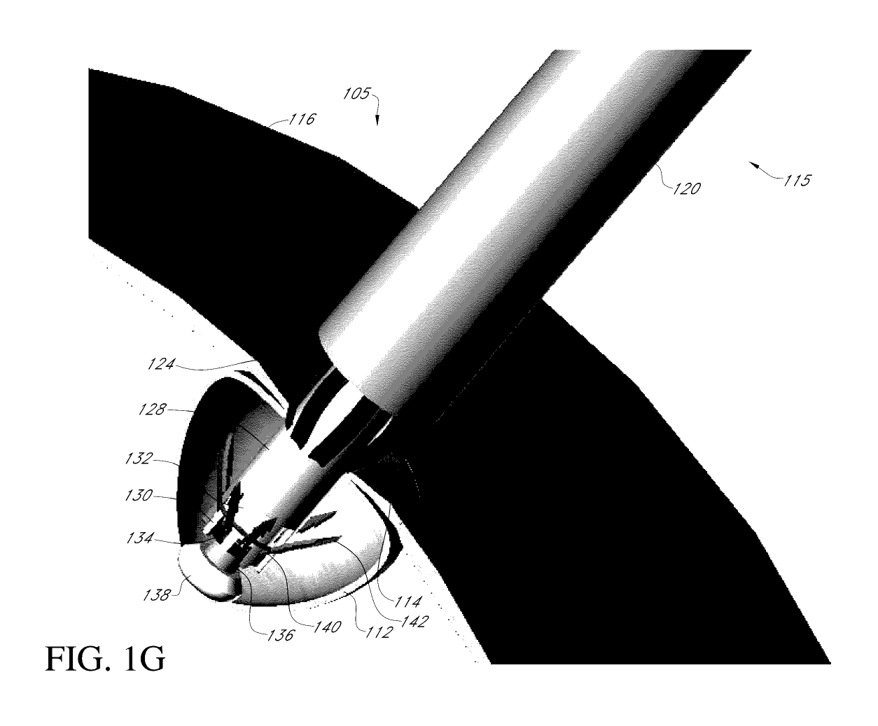

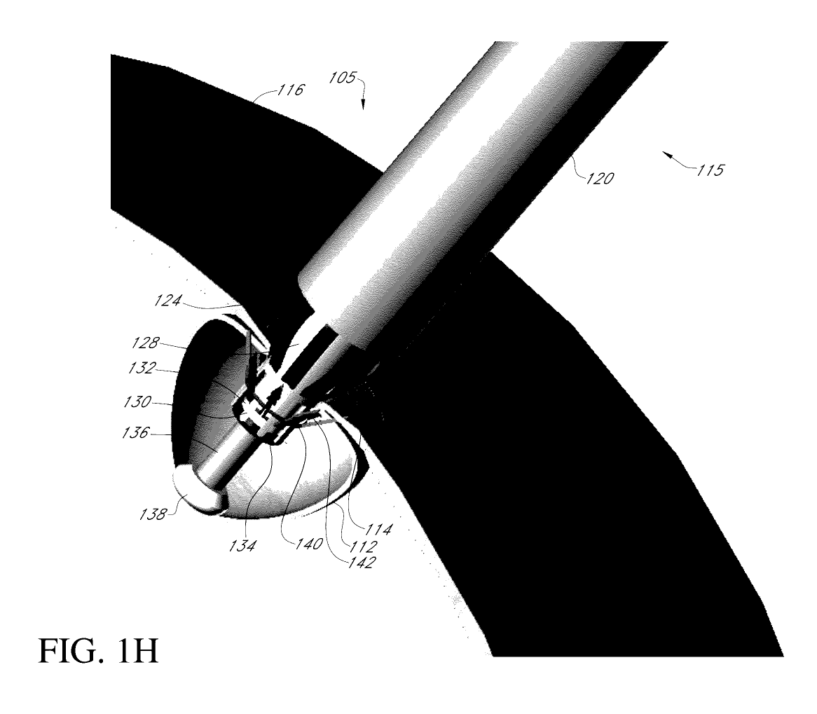

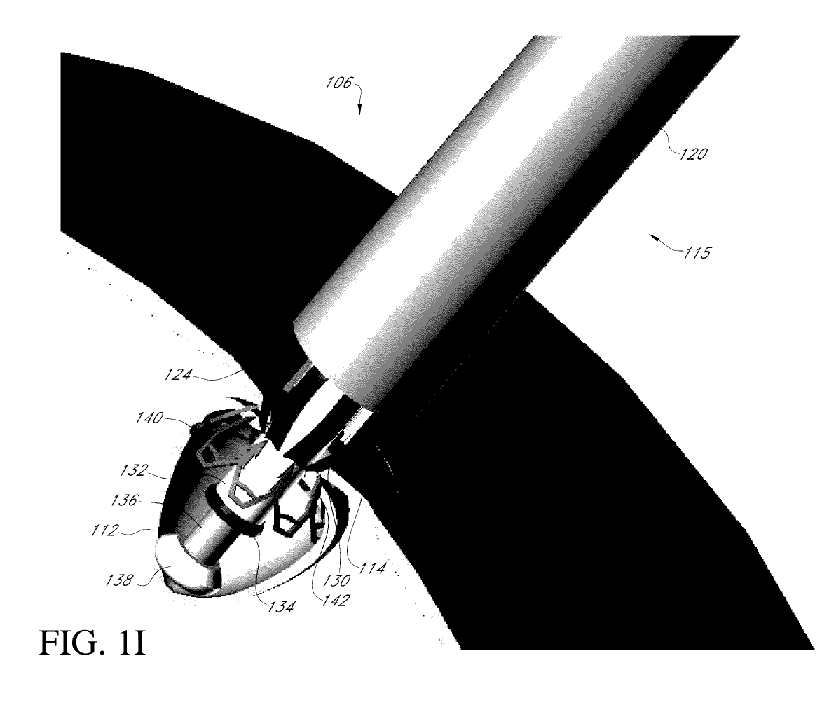

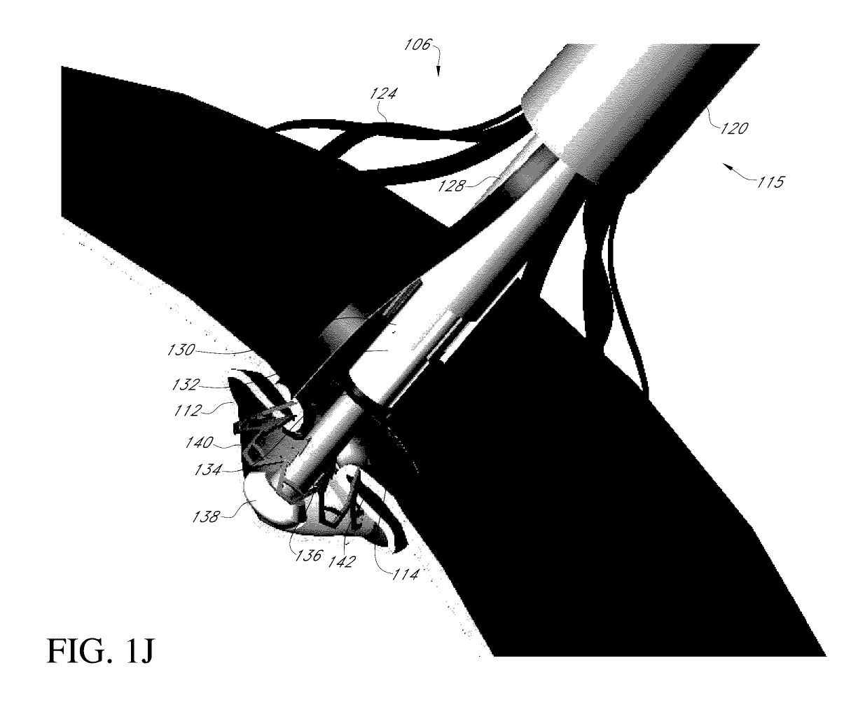

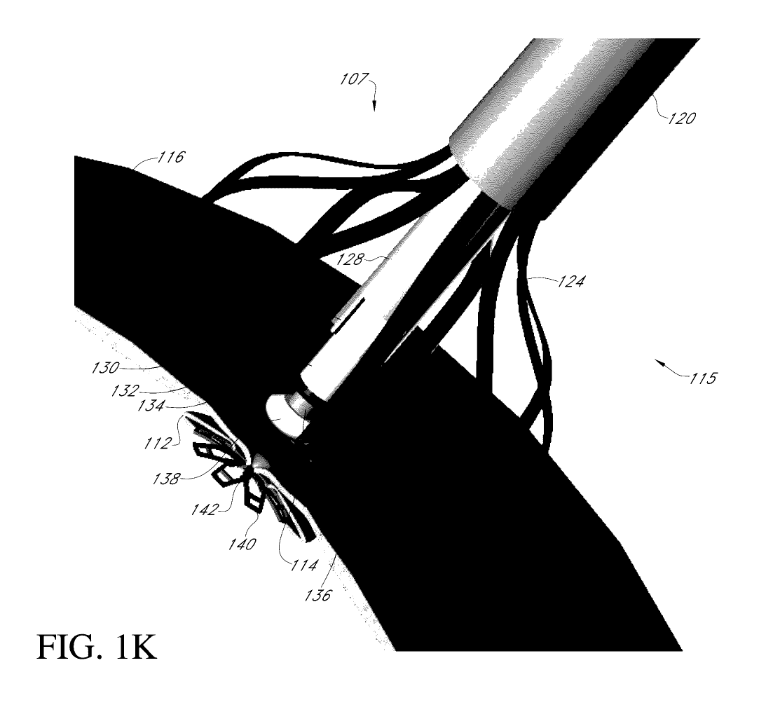

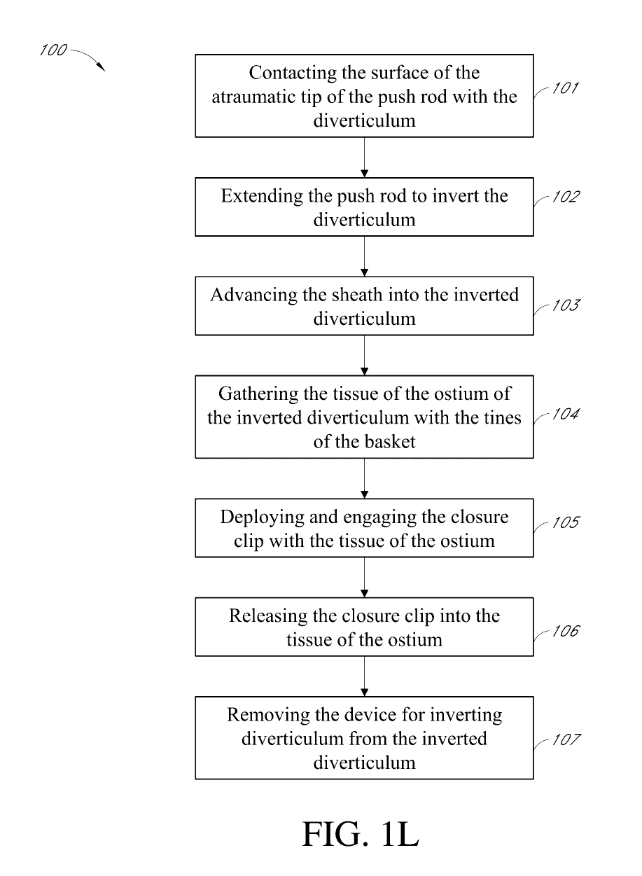

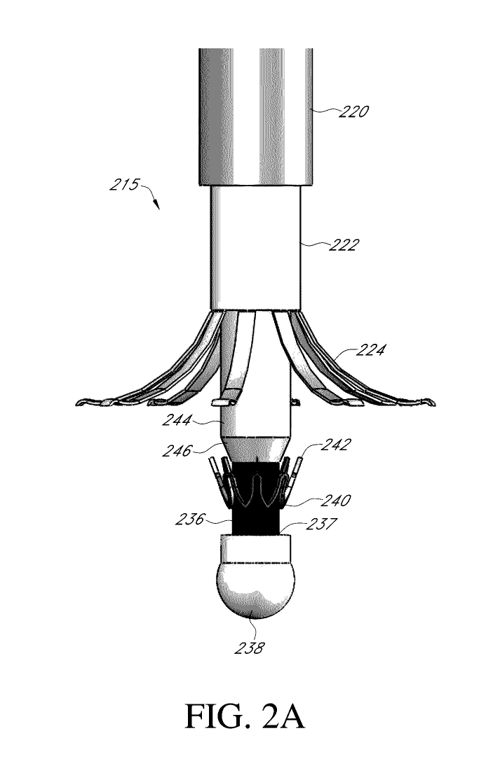

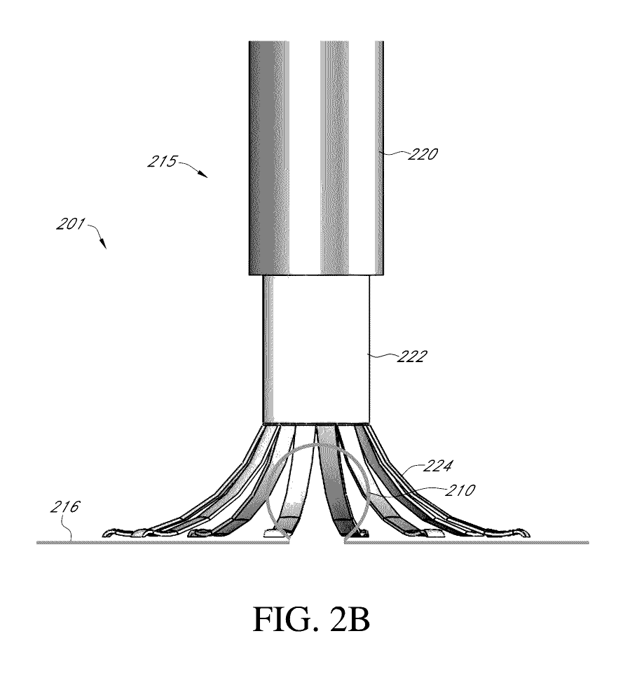

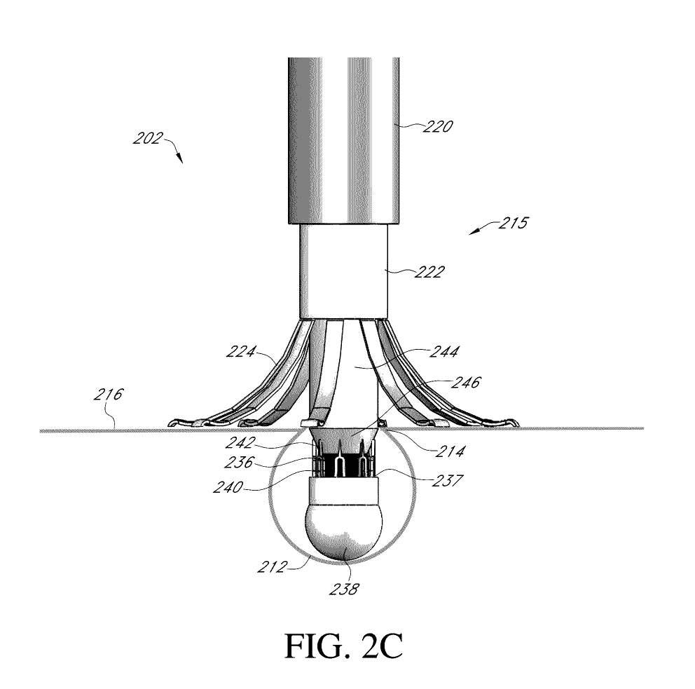

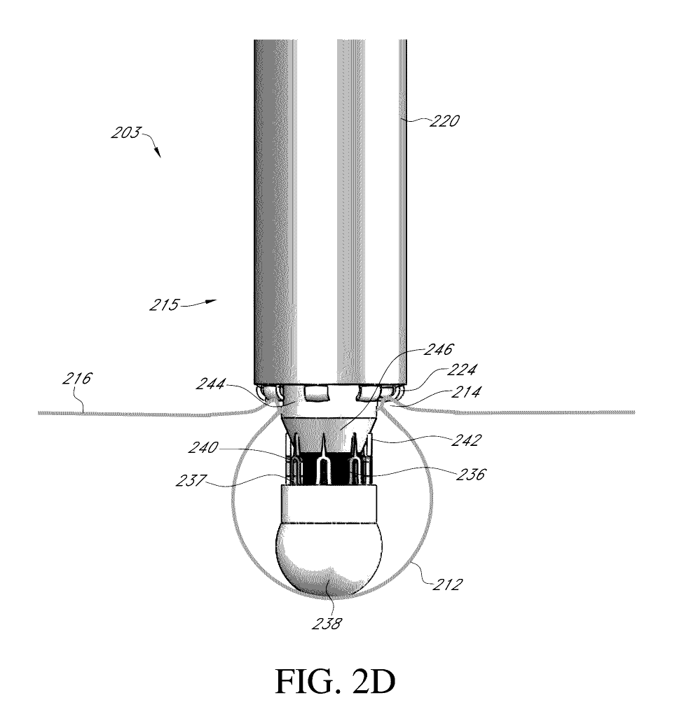

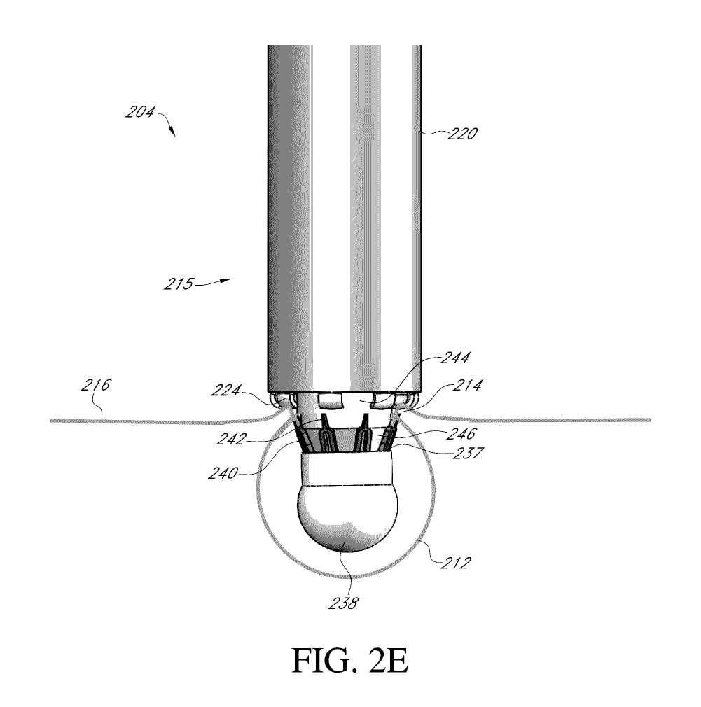

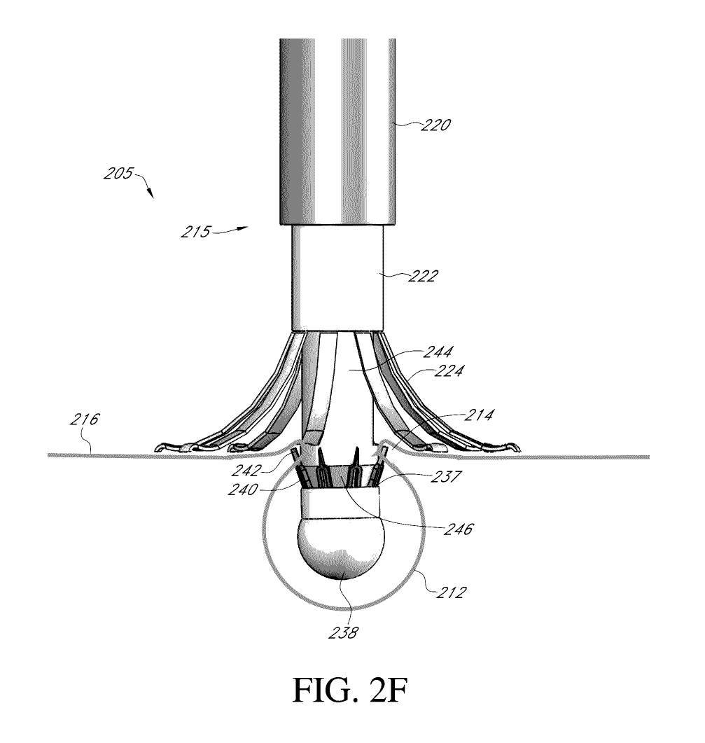

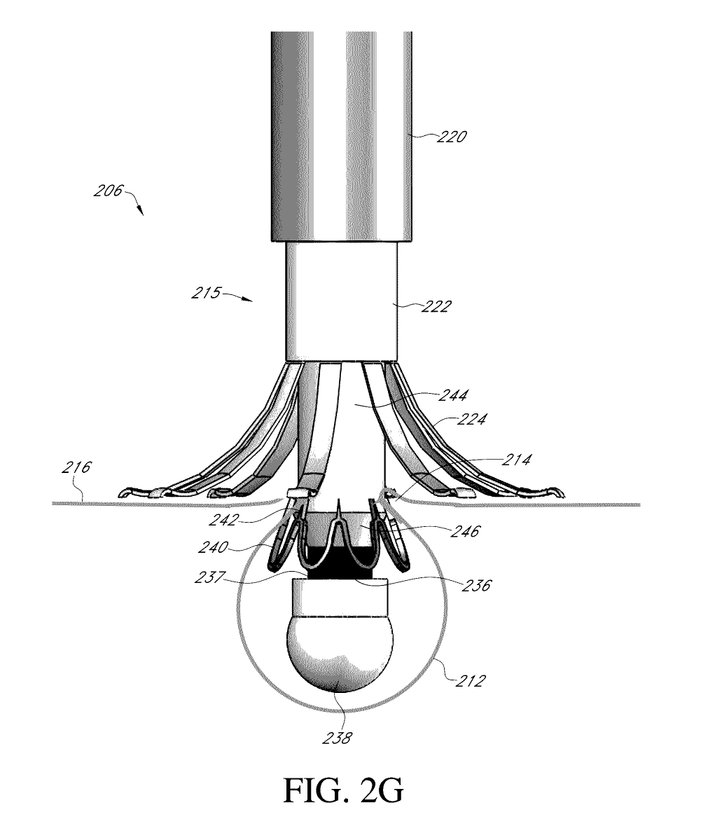

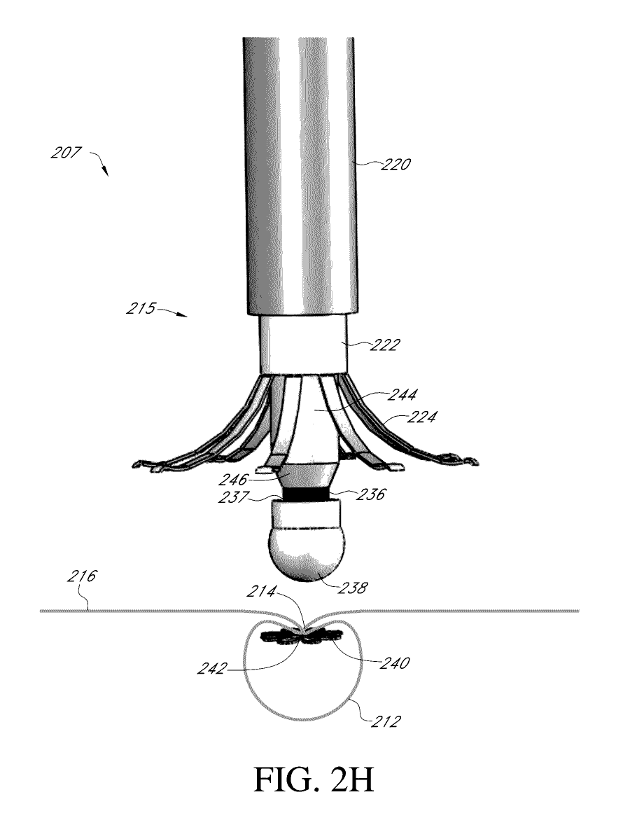

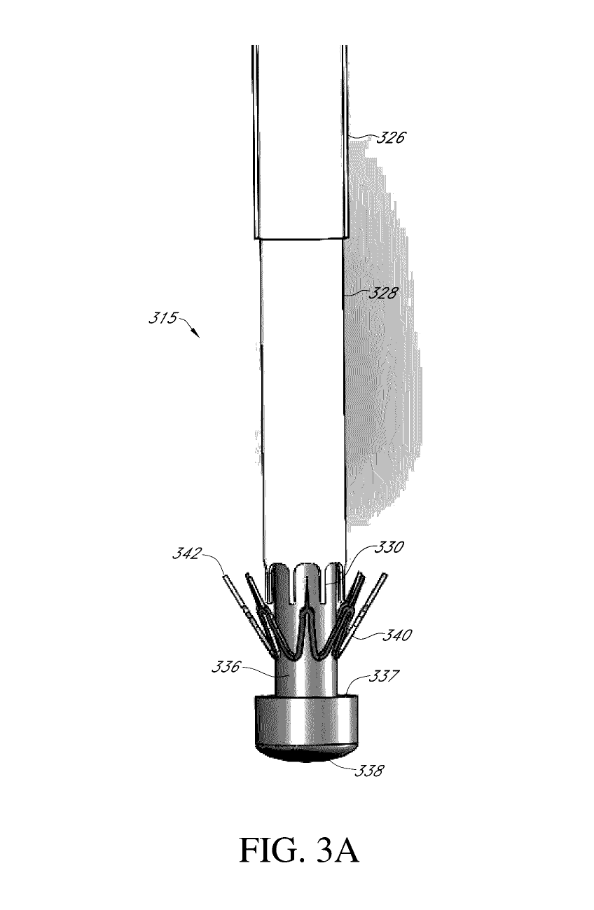

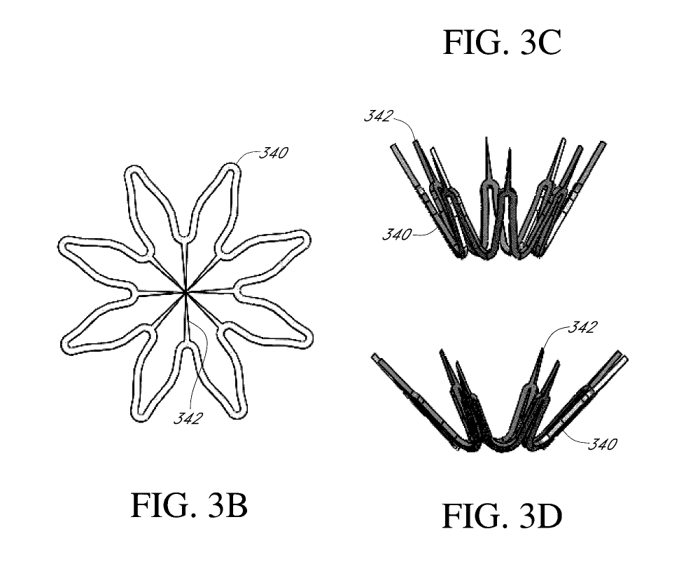

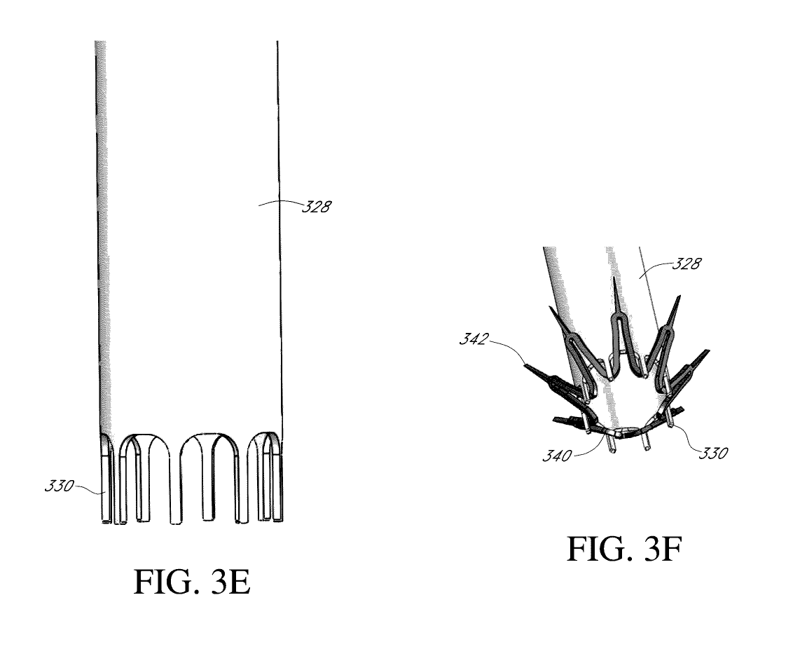

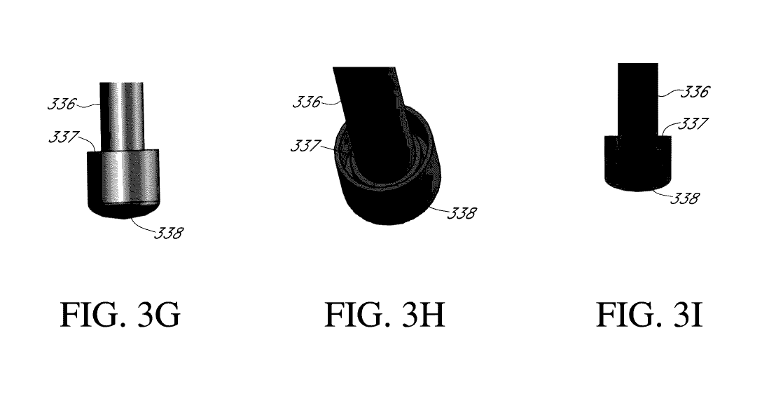

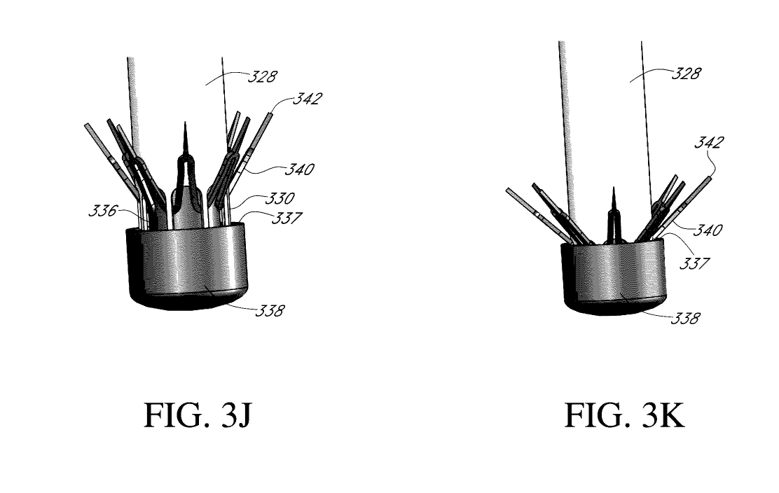

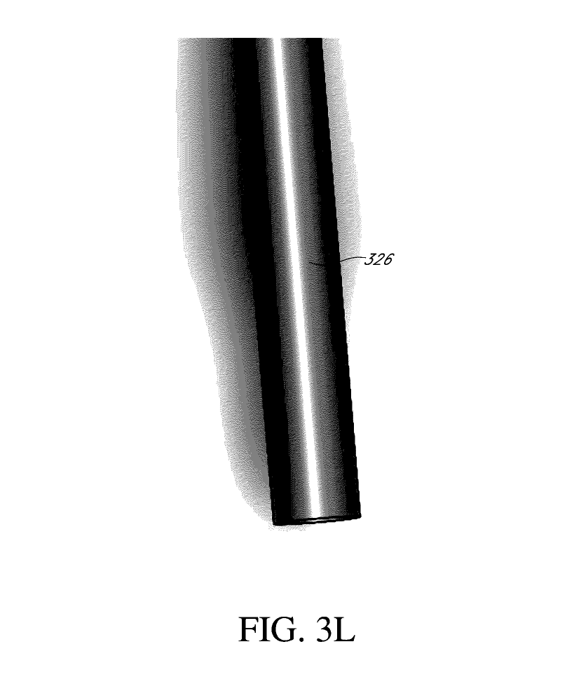

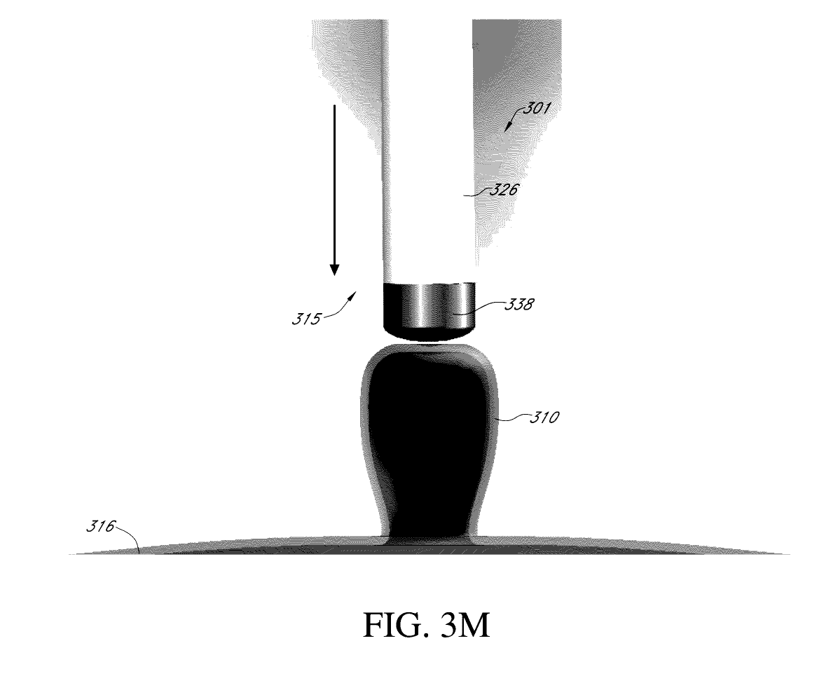

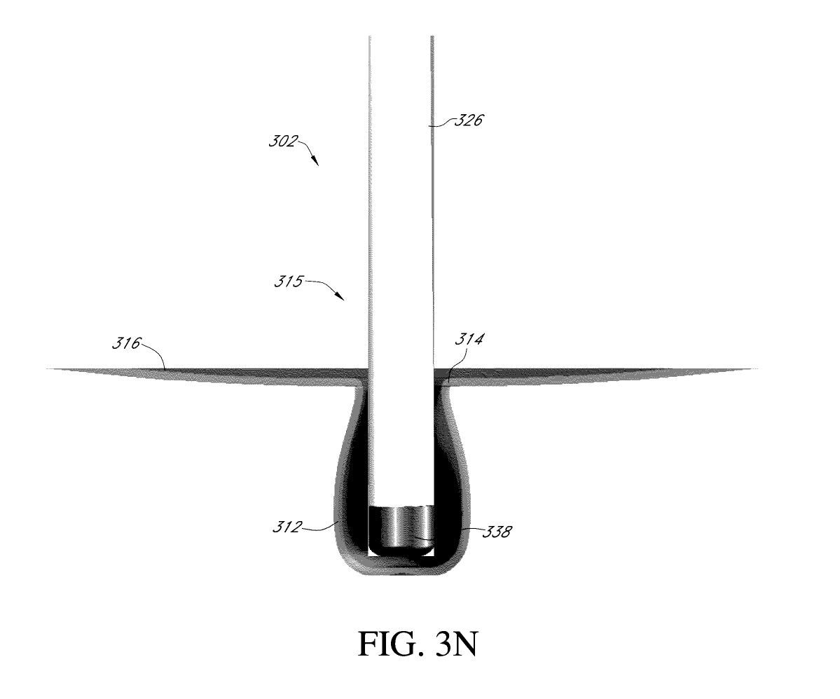

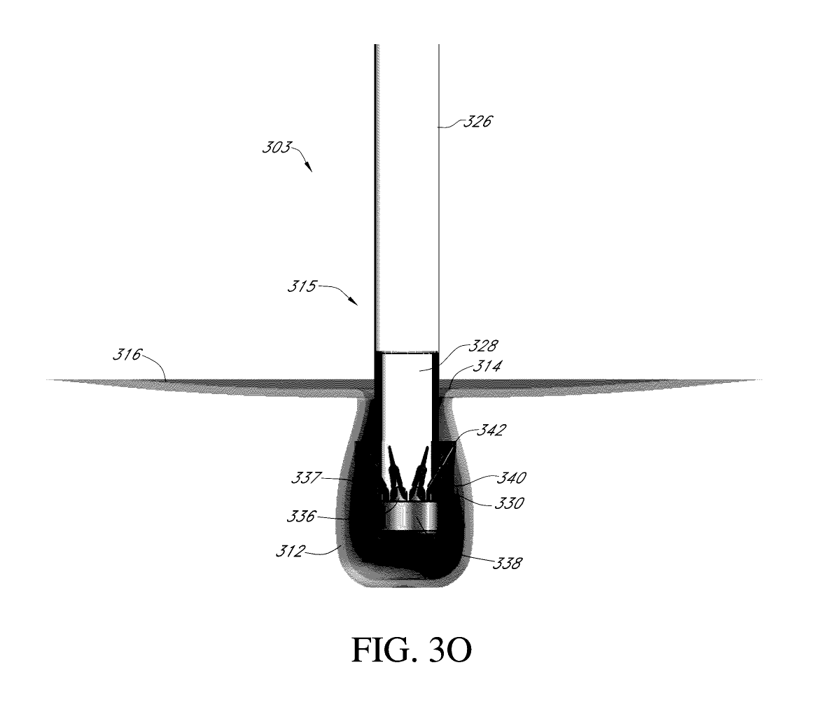

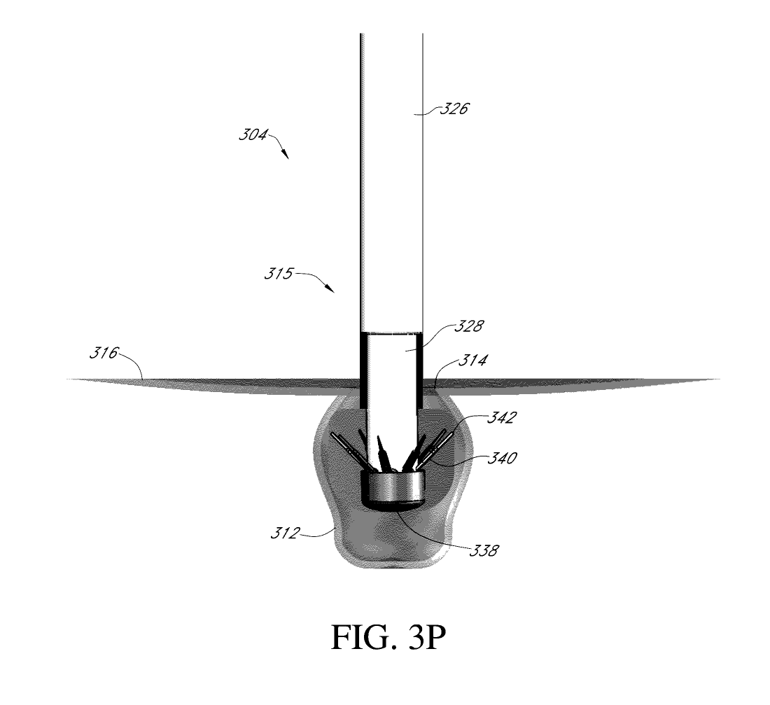

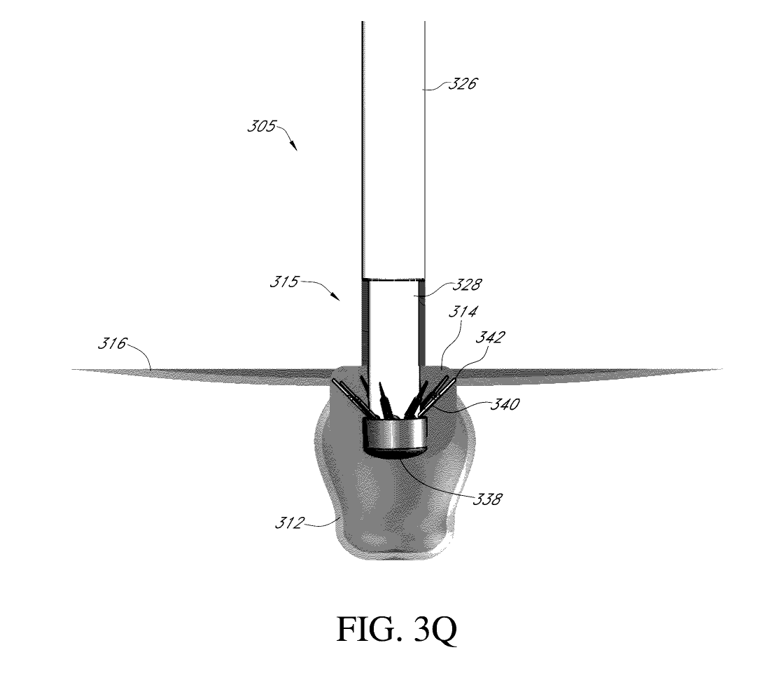

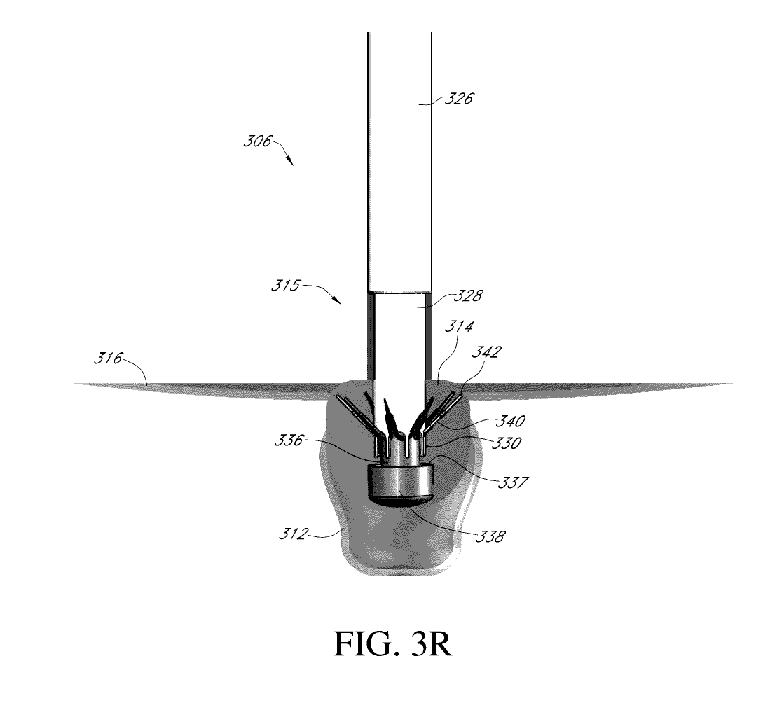

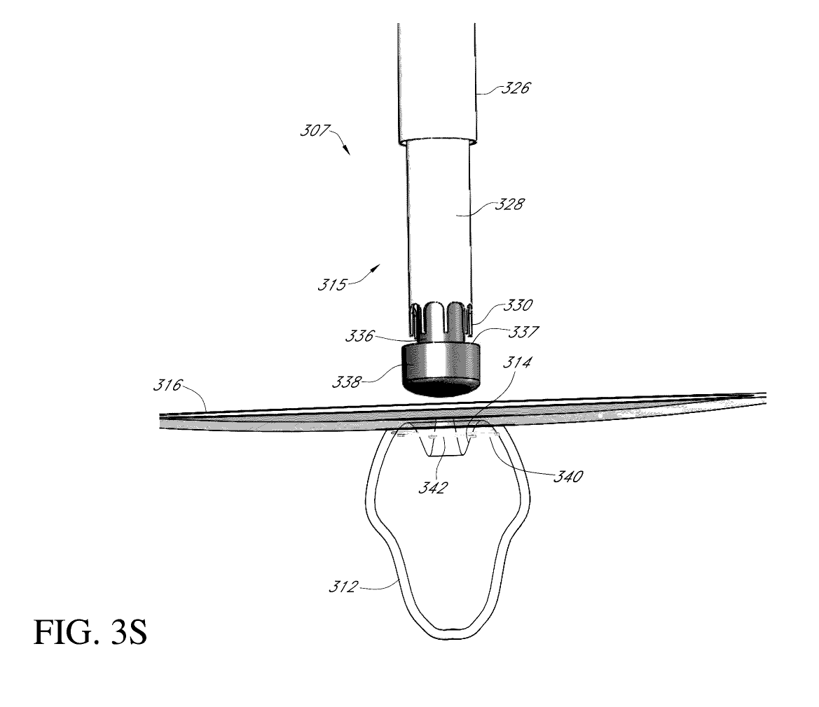

Disclosed herein are methods and devices for treating diverticular disease. FIGS. 1A-G, 2A-H, and 3A-R illustrate a plurality of views of a device for inverting diverticulum in a method for treating diverticulum disease. FIGS. 1A-G illustrate a plurality of views of embodiments of a device for inverting diverticulum 115 in an embodiment of a method for inverting diverticula 100. FIGS. 2A-H illustrate a plurality of views of another embodiment of a device for inverting diverticulum 215 in some embodiments of a method of inverting diverticula. Finally, FIGS. 3A-R illustrate a plurality of view of another embodiment of a device for inverting diverticulum 315 in some embodiments of a method of inverting diverticula.

Turning first to the embodiment of a device for inverting diverticulum 115 illustrated in FIG. 1A, in some embodiments the device for inverting diverticulum 115 is composed of a plurality of components that are disposed coaxially about each other. As will be illustrated in the method of inverting diverticula 100, each of the components of the device for inverting diverticulum 115 are configured to be moveable relative to each other.

In some embodiments the device for inverting diverticulum 115 can include a closer 120, a basket shaft 122, a sheath 126, a clip tube 128, a tube stop 132, and a push rod 136. In some embodiments, the basket shaft 122 can further include a basket 124 at the distal end. As well, in some examples, the clip tube 128 can include a section including locking structures 130 at the distal end. As will be discussed, the locking structures 130 of the clip tube 128 can help to retain a closure clip 140. In some embodiments, the push rod 136 can further include an atraumatic tip 138 at the distal end. Each of the tubular components of the device for inverting diverticulum 115 can be composed of hardened steel.

As seen in FIG. 1A, the push rod 136 is located at the center of the device for inverting diverticulum 115. In some embodiments, the push rod 136 includes an atraumatic tip 138 at the distal end. As will be discussed below, the atraumatic tip 138 of the push rod 136 can serve to invert a target diverticulum during the method of inverting diverticula 100.

In some examples, a tube stop 132 is disposed coaxially about the push rod 136. In some variants, a flange 134 is located at the distal end of the tube stop 132 such that the flange 134 forms a wider diameter than the distal opening of the tube stop 132. The push rod 136 and the tube stop 132 can move relative to each other such that the push rod 136 can be withdrawn into the tube stop 132. In some examples, the atraumatic tip 138 has a sufficiently wide diameter such that it prevents the push rod 136 from being withdrawn entirely into the tube stop 132. As will be discussed in more detail below, the tube stop 132—in particular the flange 134 of the tube stop 132—can help to adjust the angle on which the closure clip 140 is retained on the clip tube 128 as well as adjust the angle that the closure clip 140 penetrates into the target diverticulum.

In some variants, a clip tube 128 is disposed about the tube stop 132. As noted above, in some examples, the clip tube 128 includes a plurality of locking structures 130 that are located at the distal end of the clip tube 128. The locking structures 130 can be configured to retain a closure clip 140 on the distal end of the device for inverting diverticulum 115. The clip tube 128 is configured such that it is moveable relative to the tube stop 132. In some examples, the locking structures 130 of the clip tube 128 can extend past the flange 134 of the tube stop 132. As will be discussed below, the locking structures 130 can help to retain the closure clip 140 on the device for inverting diverticulum 115. In some variants, along with the flange 134, the locking structures 130 can adjust the angle of the closure clip 140 on the distal end of the device for inverting diverticulum 115 to better allow the closure clip 140 to engage with the tissue of the target inverted diverticulum. As well, the interaction of the flange 134 and the locking structures 130 can also allow the closure clip 140 to be deployed into the inverted diverticulum.

In some embodiments, a sheath 126 can be disposed about the clip tube 128. In some examples, the diameter of the sheath 126 should be wide enough such that it can accommodate the clip tube 128 and the attached closure clip 140. In some examples, the purpose of the sheath 126 is to prevent the closure clip 140 or the locking structures 130 of the clip tube 128 from catching onto anything prior to deployment or placed into the inverted diverticulum.

In some examples, the device for inverting diverticulum 115 can further include a basket shaft 122. As illustrated in FIG. 1A, in some variants, the basket shaft 122 can include a basket 124 located at the distal end of the basket shaft 122. As the basket shaft 122 is configured to be moveable relative to the sheath 126, the basket shaft 122 can be extended or withdrawn proximal and/or distal to the other components of the device for inverting diverticulum 115. As will be described in more detail below, the basket 124 is configured to retain the tissue about the ostium of the inverted diverticulum. This can help to better deploy the closure clip 140 into the target tissue.

Lastly, in some embodiments, the device for inverting diverticulum 115 can further include a closer 120. In some examples, the closer 120 is configured to be disposed about the internal components of the device for inverting diverticulum 115. As the closer 120 is moveable relative to the basket shaft 122, the closer 120 can extend distally to cover and retain the basket 124 of the basket shaft 122. In some examples this can help to maintain the minimal profile of the device for inverting diverticulum 115 prior to use of the device in the method of inverting diverticula 100.

As noted above, the various components of the device for inverting diverticulum 115 can be moveable relative to each other. As well, in order to maintain a minimal profile of the device for inverting diverticulum 115 prior to use of the device for inverting diverticulum 115 in the method of inverting diverticula 100, the closer 120 can be used to retain the basket 124 of the basket shaft 122. Similarly, to prevent the inadvertent deployment or interaction of the closure clip 140 with the surrounding environment, the sheath 126 can be disposed about the clip tube 128, tube stop 132, and the closure clip 140 retained in between. FIG. 1B illustrates the initial configuration of the device for inverting diverticulum 115 after the basket 124 is released from the closer 120. As described, the sheath 126 is disposed about the clip tube 128, tube stop 132, and the closure clip 140.

Once the device for inverting diverticulum 115 has been inserted into the body, the device can be used to treat a diverticulum. FIG. 1L illustrates a flow chart of the method of inverting diverticula 100. Each of the steps of the method of inverting diverticula 100 are illustrated in FIGS. 1C-K. In the method of inverting diverticula 100, the device for inverting diverticulum 115 is used to treat a diverticulum 110 that is located on the surface of the outer wall of colon 116

FIG. 1C illustrates step 101 in the method of inverting diverticula 100. As illustrated, the outer wall of colon 116 includes a diverticulum 110 that protrudes from the diverticulum 110. In some embodiments, in step 101, the atraumatic tip 138 of the push rod 136 is extended to contact the top surface of the diverticulum 110. In some examples, the atraumatic tip 138 enters substantially perpendicular to the colon surface. Initial contact adjustments may be necessary in order to evenly contact the diverticulum 110.

Next, as illustrated in FIG. 1D, the diverticulum 110 is inverted at step 102. In some embodiments, as can be seen, the push rod 136 can extend distally such that the atraumatic tip 138 compresses the diverticulum 110 to force it to invert. As the diverticulum 110 inverts, the outer lip of the tissue of the diverticulum 110 can form an ostium that can provide the closure clip 140 with tissue to engage with.

Once the diverticulum 110 has been inverted, as illustrated in FIG. 1E, step 103 involves fully forming the inverted diverticulum 112 and preparing the basket 124 to engage with the surrounding tissue of the ostium 114. As can be seen, after the diverticulum 110 is inverted with the atraumatic tip 138 of the push rod 136, the sheath 126 with the retained clip tube 128, tube stop 132, and closure clip 140 can be further advanced into the inverted diverticulum 112. The advancing of the device into the inverted diverticulum 112 better allows the tines of the basket 124 to engage with the ostium tissue 114 in tension (not pictured in FIG. 1E). In some examples, prior to the basket 124 touching the tissue of the ostium 114, the diameter of the basket 124 can be adjusted to 2-3 mm greater than the ostium 114. In some embodiments, once the basket 124 engages with the ostium 114 tissue, the outer wall of colon 116 is depressed by approximately 1 inch.

Next, FIG. 1F illustrates step 104 which describes tissue acquisition by the basket 124 once the device for inverting diverticulum 115 is in position. As illustrated, while the tissue of the inverted diverticulum 112 and ostium 114 are still under tension by the inserted distal end of the device for inverting diverticulum 115, the basket 124 is closed about the ostium 114 to draw up the tissue evenly. In some embodiments, this is accomplished by advancing the closer 120 distally past the tines of the basket 124. As the closer 120 is advanced, the tines of the basket 124 are drawn together to capture the tissue of the ostium 114. In some embodiments, if the tines of the basket 124 slip or the creases of the captured tissue are largely asymmetrical, the basket 124 can be opened and step 104 can be repeated.

Once the tissue of the ostium 114 has been captured by the basket 124, step 105 as illustrated in FIGS. 1G-H disclose deploying and engaging of the closure clip 140 with the tissue of the ostium 114 while located in the inverted diverticulum 112. As illustrated in FIG. 1G, the inverted diverticulum 112 can be expanded (e.g. with gas) to allow the closure clip 140 to expand. As described above, the attached closure clip 140 can be expanded to allow the clip tines 142 to flare outwards by withdrawing the sheath 126 in a proximal direction. In some embodiments, the withdrawn sheath 126 allows the clip tines 142 of the closure clip 140 to flare outwards. In some embodiments, as illustrated in FIG. 1H, the clip tube 128 and the tube stop 132 are withdrawn proximally to engage with the tissue of the inverted diverticulum 112 and ostium 114. As discussed, in some examples, because the inverted diverticulum 112 is expanded with gas, this prevents the inverted diverticulum 112 tissue from bunching up over the tines. As illustrated, the clip tines 142 of the closure clip 140 are targeted to penetrate into the outer wall of colon 116.

In some examples, once the closure clip 140 has penetrated into the tissue, the method of inverting diverticula 100 can further include step 106 which illustrates the releasing of the closure clip 140 into the tissue. As illustrated in FIG. 1I, the inverted diverticulum 112 is longer inflated (e.g. the gas can be stopped). To release the closure clip 140 into the target tissue, the clip tube 128 can first be withdrawn proximally. In some examples, the proximal withdrawing of the locking structures 130 releases the closure clip 140 from the angle on the clip tube 128 and allows the closure clip 140 to begin to rotate into its final position. The tube stop 132 is not withdrawn so as to maintain the closure clip 140 on the device. Next, as illustrated in FIG. 1J, after the clip has rotated into its final position (e.g. planar position), the tube stop 132 is withdrawn in a proximal direction. In some examples, the tube stop 132 and the flange 134 slips through the center of the inserted closure clip 140. As well, as illustrated in FIG. 1J, the basket 124 can be opened to release the tissue of the ostium 114. In some embodiments (not pictured), the closure clip 140 can be sprung to the clip tube 128 such that the withdrawal of the tube stop 132 and flange 134 can be self-driven.

Finally, the device for inverting diverticulum 115 can be removed from the inverted diverticulum 112 in step 107 as illustrated in FIG. 1K. To remove the device for inverting diverticulum 115 from the inverted diverticulum 112, the push rod 136 and atraumatic tip 138 can be withdrawn gently to pull it past the implanted closure clip 140. As seen in FIG. 1K, once the push rod 136 and atraumatic tip 138 are removed from the inverted diverticulum 112, the closure clip 140 is allowed to rotate such that it is fully flattened. In some examples, as the closure clip 140 flattens, the clip tines 142 of the closure clip 140 capture the tissue of the ostium 114 so as to flatten and secure the inverted diverticulum 112 closed on the outer wall of colon 116.

FIGS. 2A-H illustrates another embodiment of the device for inverting diverticulum 215. FIG. 2A illustrates a side perspective of the distal end of the device for inverting diverticulum 215.

Turning now to another embodiment of a device for inverting diverticulum 215, in some embodiments the device for inverting diverticulum 215 is composed of a plurality of components that are disposed coaxially about each other. Similar to the method of inverting diverticula 100, in the method of inverting diverticula as illustrated in FIGS. 2B-H, each of the components of the device for inverting diverticulum 215 are configured to be moveable relative to each other.

In some embodiments, the device for inverting diverticulum 215 can include a closer 220, a basket shaft 222, a ramp tube 244, and a push rod 236. As can be seen, the device for inverting diverticulum 215 is largely similar to the device for inverting diverticulum 115 with a few adjustments. For example, in some embodiments, the device for inverting diverticulum 215 includes a ramp tube 244 that can further include a ramped portion 246 at the distal end of ramp tube 244. Unlike the closure clip 140 in the device for inverting diverticulum 115 that is attached to the locking structures 130 of the clip tube 128, the closure clip 240 is disposed about the push rod 236. In some embodiments, the atraumatic tip 238 of the device for inverting diverticulum 215 can further include an inner opening 237. Each of the tubular components of the device for inverting diverticulum 115 can be composed of hardened steel.

As illustrated in FIG. 2A, the push rod 236 is located at the center of the device for inverting diverticulum 215. In some embodiments, the push rod 236 includes an atraumatic tip 238 at the distal end. In some examples, at the connection point between the push rod 236 and the atraumatic tip 238, the outer surface of the push rod 236 and the inner surface of the atraumatic tip 238 can form an inner opening 237. In some examples, the inner opening 237 can be configured to accommodate a portion of the closure clip 240 to adjust the angle that the closure clip 240 is located on the push rod 236. In other embodiments, the inner opening 237 of the atraumatic tip 238 can help to keep the closure clip 240 in a “safe” position prior to delivery. As will be discussed below, when it is ready to deliver the closure clip 240, the atraumatic tip 238 can be withdrawn to guide the closure clip 240 into the ramped position.

In some examples, the ramp tube 244 is disposed coaxially about the push rod 236. In some variants, the ramp tube 244 includes a ramped portion 246 that is located at the distal end of the ramped portion 246. The ramp tube 244 can be moveable relative to the push rod 236. In some examples, the ramped portion 246 of the ramp tube 244 can move an attached closure clip 240 into the inner opening 237 of the atraumatic tip 238 to alter the angle of the closure clip 240. The ramped portion 246 can help to adjust the angle on which the closure clip 240 is retained on the push rod 236 as well as to adjust the angle that the closure clip 240 penetrates into the target diverticulum. The ramped portion 246 can flare the clip into delivery position.

In some variants, the basket shaft 222 can be disposed about the ramp tube 244. In some examples, the basket shaft 222 can include a basket 224 located at the distal end of the basket shaft 222. As the basket shaft 222 is configured to be moveable relative to the ramp tube 244, the basket shaft 222 can be extended or withdrawn proximal and/or distal to the other components of the device for inverting the diverticulum 215. As will be described in more detail below, the basket 224 can be configured to retain the tissue about the ostium of the inverted diverticulum. This can help to better deploy the closure clip 240 into the target tissue.

In some embodiments, the device for inverting diverticulum 215 can further include a closer 220. In some examples, the closer 220 is configured to be disposed about the internal components of the device for inverting diverticulum 215. As the closer 220 is moveable relative to the basket shaft 222, the closer 220 can extend distally to cover and retain the basket 224 of the basket shaft 222. In some examples, this can help to maintain the minimal profile of the device for inverting diverticulum 215 prior to use of the device in the method of inverting diverticula.

The device for inverting diverticulum 215 can be used to treat a diverticulum. FIGS. 2B-H illustrate one embodiment of the method of inverting diverticula. The method of inverting diverticula is generally similar to the method of inverting diverticula 100.

FIG. 2B illustrates step 201 in the method of inverting diverticula. As illustrated, in some embodiments, after the device for inverting diverticulum 215 is inserted through the trocar, the closer 220 can be withdrawn in a proximal direction to release the 22 and the basket 224. The device for inverting diverticulum 215 can then be advanced until the basket 224 is centered on the target diverticulum 210.

Next, at FIG. 2C, the method of inverting diverticula can include step 202 wherein the atraumatic tip 238 on the push rod 236 can be advanced in a distal direction to invert the diverticulum 210. In some embodiments, the distal end of the atraumatic tip 238 can be spring loaded to prevent excessive force from being placed on the diverticulum 210 and in order to accommodate diverticulum 210 of different sizes. In some examples, at step 202, the closure clip 240 is retracted into the inner opening 237 of the atraumatic tip 238. In this configuration, the clip tines 242 is in a “safe” position, such that the clip tines 242 do not interfere with any tissue as it is inserted into the inverted diverticulum 112. In some examples, the inverted diverticulum 212 can be inflated (e.g. with gas) to allow better presentation of the ostium 214 to the closure clip 240.

In some embodiments, the method of inverting diverticula can include step 203 illustrated in FIG. 2D. Here, the basket 224 of the basket shaft 222 is closed around the tissue of the ostium 214 formed from the inverted diverticulum 212 on the outer wall of colon 216. In some examples, the basket 224 can grab the tissue by advancing the closer 220 in a distal direction until it completely covers the length of the basket 224. This can allow the basket 224 to pull and secure the healthy tissue of the ostium 214 around the ramp tube 244.

As illustrated in FIG. 2E, the method of inverting diverticula can include step 204 wherein the closure clip 240 is ramped into a “deploy” position. In some examples, at step 204, the push rod 236 is withdrawn in a distal direction with respect to the ramp tube 244. The ramped portion 246 of the ramp tube 244 can guide the closure clip 240 into the “deploy” position. In some examples, in the “deploy” position, the clip tines 242 of the closure clip 240 are flared outwards and engage with the gathered tissue at the ostium 214 of the inverted diverticulum 212. In some embodiments (not illustrated) the closure clip 240 can be released by continuing to withdraw the atraumatic tip 238 in a proximal direction. While the inner ledge 237 holds the clip in place, the movement of the atraumatic tip 238 is under the clip and ejects the closure clip 240 into the tissue as the closure clip 240 is moved along the ramped portion 246 of the ramp tube 244. Once the closure clip 240 is free of the inner ledge 237, it can begin to expand outwardly to return to its original planar shape.

Once the clip tines 242 of the closure clip 240 has been inserted into the tissue of the ostium 214, the method of inverting diverticula can proceed to step 205. As illustrated in FIG. 2F, the closer 220 can be withdrawn in a distal direction. As the closer 220 is withdrawn, the basket 224 can be opened—thereby releasing the gathered tissue of the ostium 214 around the neck of the inverted diverticulum 212.

In some embodiments, the method of inverting diverticula can then include step 206 as illustrated in FIG. 2G. Once the clip tines 242 of the closure clip 240 are secured in the tissue of the ostium 214, the push rod 136 can be advanced in a distal direction. In doing so, the closure clip 240 is released from the inner opening 237 of the atraumatic tip 238. This can allow the closure clip 240 to rotate with the clip tines 242 in gripping tissue around the ramp tube 244.

Once the closure clip 240 has been released from the inner opening 237 of the atraumatic tip 238, the method of inverting diverticula can proceed to step 207 where the device for inverting diverticulum 215 is retracted from the inverted diverticulum 112. In some embodiments, once the closure clip 240 has been released, the ramp tube 244, the ramped portion 246 of the ramp tube 244, the push rod 236, and the atraumatic tip 238 of the push rod 236 can be retracted through the ostium 214 of the inverted diverticulum 112. In some examples, once the distal end of the device for inverting diverticulum 215 has been fully retracted, the closure clip 240 can closed to its natural flat shape and grip the healthy tissue of the ostium 214 together with the clip tines 242.

Finally, FIGS. 3A-S illustrate another embodiment of the device for inverting diverticulum 315. FIG. 3A illustrates a side perspective of the distal end of the device for inverting diverticulum 315. FIGS. 3B-L illustrate a plurality of views of the components in the device for inverting diverticulum 315. Many of the illustrations of the components of the device for inverting diverticulum 315 can be similarly applicable to the device for inverting diverticulum 115 and device for inverting diverticulum 215 discussed above.

The device for inverting diverticulum 315 has elements that resemble or are similar to the device for inverting diverticulum 115 and device for inverting diverticulum 215 described above. Accordingly, numerals used to identify features of the device for inverting diverticulum 115 and device for inverting diverticulum 215 are incremented by a factor of one hundred to identify like features of the device for inverting diverticulum 315. This numbering conventional generally applies to the remainder of the figures. Any component or step disclosed in any embodiment in this specification can be used in other embodiments.

In some embodiments, the device for inverting diverticulum 315 (as illustrated in FIG. 3A), can be composed of a plurality of components that are disposed coaxially about each other. As will be illustrated in the method of inverting diverticula, each of the components of the device for inverting diverticulum 315 are configured to be moveable relative to each other. Unlike the previous two embodiments, in some examples, the device for inverting diverticulum 315 does not include a basket for securing the ostium of the inverted diverticulum. As discussed above, in some embodiments, each of the tubular components of the device for inverting diverticulum 115 can be composed of hardened steel.

As seen in FIG. 3A, the push rod 336 is located at the center of the device for inverting diverticulum 315. As can be seen, in some embodiments, the closure clip 340 is disposed about the push rod 336 of the device. In some embodiments, the push rod 336 can include an atraumatic tip 338 at the distal end.

FIGS. 3B-D illustrate a plurality of views of the closure clip 340. Each of these illustrations can be applicable to any discussion of the closure clip provided above. FIG. 3B illustrates a top view of an embodiment of the closure clip 340 in its relaxed state. In its relaxed state, the closure clip 340 is flat with the clip tines 342 pointing inward. In some embodiments, this is the form the closure clip 340 will take after it has been delivered inside of the inverted diverticulum 112. In some embodiments, the closure clip 340 can have tines that are 1.88 mm. FIG. 3C illustrates the closure clip 340 as it is loaded on the locking structures 330 of the clip tube 328. In this loaded state, the closure clip 340 is in a semi-flared shape. Lastly, FIG. 3D illustrates the closure clip 340 as it is flared even more for delivery to capture the tissue in the ostium 314. As was discussed, and will be discussed below, in the various embodiments of the method of inverting diverticula, the flaring of the clip tines 342 provide the closure clip 340 with a broader reach to engage the surrounding tissue of the ostium 314.

In some examples, at the connection point between the push rod 336 and the atraumatic tip 338, the outer surface of the push rod 336 and the inner surface of the atraumatic tip 338 can form an inner opening 337. In some examples, the inner opening 337 can be configured to accommodate a portion of the closure clip 340 to adjust the angle that the closure clip 340 is located on the push rod 336. FIGS. 3G-I illustrate a side, top, and cross-sectional view of the atraumatic tip 338. As is illustrated, the inner opening 337 provided between the outer surface of the push rod 336 and the inner surface of the atraumatic tip 338 provides an opening to accommodate a portion of the closure clip 340. As well, as illustrated in FIG. 3H, in some embodiments, the inner opening 337 can include a ledge that is adjacent the outer surface of the push rod 336.

In some examples, a clip tube 328 can be disposed about the push rod 336. In some embodiments, the clip tube 328 includes locking structures 330 at the distal end of the clip tube 328. As will be discussed in more detail below, the locking structures 330 can engage with the closure clip 340 that is disposed about the push rod 336 to secure the closure clip 340 on the distal end of the device for inverting diverticulum 315. As noted above, because the clip tube 328 and the push rod 336 are moveable relative to each other, withdrawing or advancing the clip tube 328 can cause the locking structures 330 to interact with the closure clip 340 to alter the angle in which the clip tines 342 are flared on the distal end of the device.

FIGS. 3E-F illustrate the clip tube 328 and a bottom perspective view of the clip tube 328 with closure clip 340 engaged. As illustrated in FIG. 3E, the locking structures 330 can include a plurality of equally spaced structures. FIG. 3F illustrate the closure clip 340 as it is engaged with the locking structures 330. As seen, each apex of the closure clip 340 is linked around the locking structures 330 to create the loaded flare shape—wherein the clip tines 342 are flared outwards and pointed in a proximal direction.

FIGS. 3J-K provide an illustration of the interaction between the locking structures 330 of the clip tube 328, the closure clip 340, and the atraumatic tip 338 of the push rod 336. FIG. 3J illustrates the closure clip 340 as it is loaded on the clip tube 328. In some examples, as the atraumatic tip 338 is moved up towards the clip tube 328, the ledge in the inner opening 337 can bump the apices of the closure clip 340. This can push them upward to cause a larger flare. FIG. 3K illustrates the distal end of the device for inverting diverticulum 315 and the configuration of the closure clip 340 during delivery. The structure of the inner opening 337 and atraumatic tip 338 allows the closure clip 340 to be pulled into the tissue without being inverted due to the force.

Lastly, in some embodiments, the device for inverting diverticulum 315 can further include a sheath 326 that can be disposed about the clip tube 328. In some examples, as seen in FIG. 3L, the sheath 326 can be wide enough such that it can accommodate the clip tube 328 and the attached closure clip 340. In some examples, the purpose of the sheath 326 is to prevent the closure clip 340 or the locking structures 330 of the clip tube 328 from unintentionally interacting with any tissue prior to deployments of the closure clip 340. The sheath 326 therefore protects the surrounding tissue as the device for inverting diverticulum 315 is inserted. As will be seen, the sheath 326 can be removed prior to deployment.

As discussed above, the device for inverting diverticulum 315 can be used to treat a diverticulum. FIGS. 3M-S illustrate another embodiment of the method of inverting diverticula. However, as will be discussed below, unlike the method of inverting diverticula discussed above, the device for inverting diverticulum 315 in the method of inverting diverticula does not include a basket for capturing the tissue of an inverted diverticula.

FIG. 3M can illustrate step 301 in the method of inverting diverticula. As is illustrated, the outer wall of colon 316 can include a diverticulum 310 that protrudes from the surface of the colon. In some embodiments, the atraumatic tip 338 located at the distal end of the device for inverting diverticulum 315 can be used to manually invert the diverticulum 310.

FIG. 3N next illustrates step 302 which shows the distal end of the device for inverting diverticulum 315 located in the inverted diverticulum 312. The distal end of the atraumatic tip 338 can further push into the inverted diverticulum 312 such that the ostium 314 is disposed snugly about the sheath 326.

The method of inverting diverticula can then include step 303 as illustrated in FIG. 3O. In some embodiments, at step 303, the sheath 326 is withdrawn in a proximal direction such that the closure clip 340 is exposed within the inverted diverticulum 312. As discussed above, the closure clip 340 at this stage is in the configuration illustrated in FIG. 3C, wherein the closure clip 340 is in a semi-flared state. In some examples, the inverted diverticulum 312 can be inflated (e.g. with gas) to allow better presentation of the ostium 214 to the closure clip 340.

In some examples, as illustrated in FIG. 3P, the method of inverting diverticula can include step 304 wherein the push rod 336 and the atraumatic tip 338 is retracted in a distal direction. As the push rod 336 is retracted, the ledge located within the atraumatic tip 338 (not pictured) can pull the closure clip 340 in a proximal direction, causing the closure clip 340 to flare to a larger diameter. In some examples, the closure clip 340 can be in the configuration illustrated in FIG. 3D wherein the flaring of the clip tines 342 can provide the closure clip 340 with a broader reach in order to engage the surrounding tissue of the ostium 314.

Once the closure clip 340 is flared outwards, the method of inverting diverticula can then include step 305 wherein the entirety of the device for inverting diverticulum 315 is retracted in a proximal direction. In some embodiments, as illustrated in FIG. 3Q, the device for inverting diverticulum 315 can be retracted in a proximal direction, this can serve to seat the clip tines 342 of the closure clip 340 into the surrounding tissue of the ostium 314. In some examples, once the clip tines 342 are in the surrounding tissue of the ostium 314, the closure clip 340 is ready to be released.

In some examples, the method of inverting diverticula can then include step 306 in order to begin the step of releasing the closure clip 340 into the tissue of the ostium 314. As illustrated in FIG. 3R, the atraumatic tip 338 of the push rod 336 is advanced in a distal direction. This can relax the closure clip 340 from the flared configuration to a semi-flared configuration. In some examples, the advancing of the atraumatic tip 338 in the inverted diverticulum 112 can allow the closure clip 340 to be released from the locking structures 330 of the clip tube 328. In some embodiments, (not illustrated) the push rod 336 can include a ramped portion. The closure clip 340 can be released by continuing to withdraw the atraumatic tip 338 in a proximal direction to cause the closure clip 340 to be ejected from the push rod 336.

Once the closure clip 340 has been released from the locking structures 330 of the clip tube 328, the distal end of the device for inverting diverticulum 315 can be retracted from the inverted diverticulum 112. As illustrated in FIG. 3S, in some embodiments, the configuration of the closure clip 340 can allow the atraumatic tip 338 to be pulled through the center of the closure clip 340. As the distal end of the device for inverting diverticulum 315 is pulled from the ostium 314 of the inverted diverticulum 312, the closure clip 340 can close around the captured tissue of the ostium 314. In some examples, the closure clip 340 can take the form of the closure clip 340 illustrated in FIG. 3B.

As the clip is being deployed, frequently the inverted diverticulum is pressure collapsed from the differential insufflation pressure being greater in the bowel than in the abdomen. As a result, the tissue snugly conforms to the clip deployment mechanism and the closure clip that is placed in the inverted diverticulum illustrated above. This can result in inconsistent implantation of the clip tines of the closure clip into the colon tissue as a result of the clip tines being unable to engage with the tissue at an advantageous angle. Because of the differential of pressures in the bowel and the abdomen, the inverted diverticulum can collapse about the closure clip and cause the clip tines to slip rather than engage.

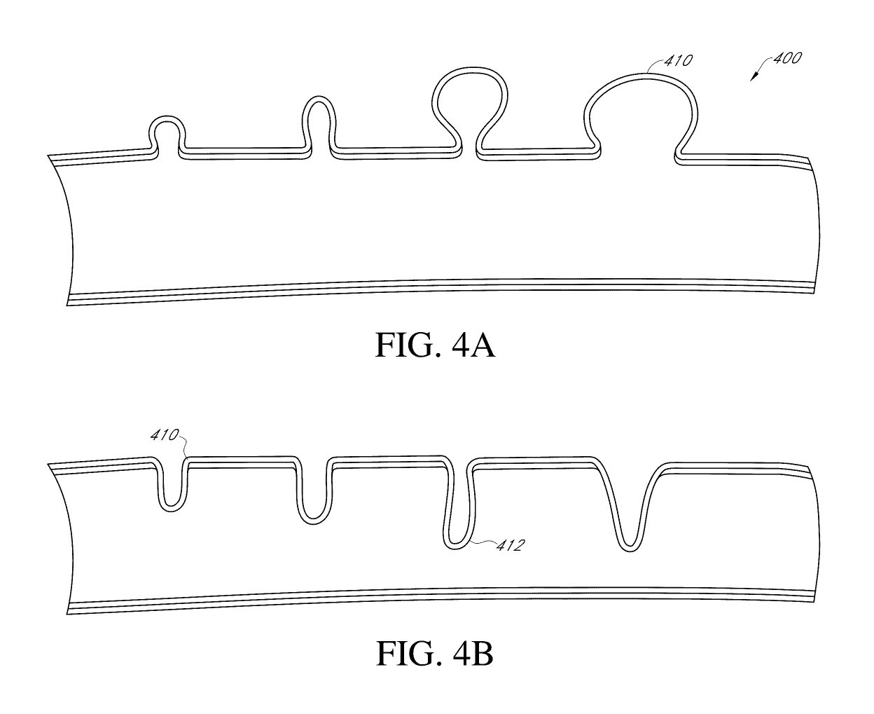

FIG. 4A-B show side views of dimensional presentations of diverticula and their inverted counterparts. As can be seen in the dimensional presentations of diverticulum 400, there are a number of different presentations and geometries of diverticulum 410 in affected patients. Similarly, these same characteristics impact the resultant shape of the inverted diverticulum 412 and the ostium 414 once the diverticulum 410 is inverted. This is due to the underlying muscle fiber orientation and inversion techniques required to manage these varying diverticulum geometries. This can be seen in FIG. 4B that illustrates the dimensional presentations of inverted diverticulum 412 and the varying configurations of the ostium 414 for each of the inverted diverticulum. The variance in ostium 414 of inverted diverticulum can have a significant impact on the ability of the clips to engage the colonic tissue in order to penetrate the wall and close around healthy tissue and provide a complete seal. Further, at each of the steps for the method of inverting diverticula 100 discussed above, variations in anatomy and presentation of anatomy can significantly impact the success of a given step in the process and therefore compromise the overall success of the procedure.

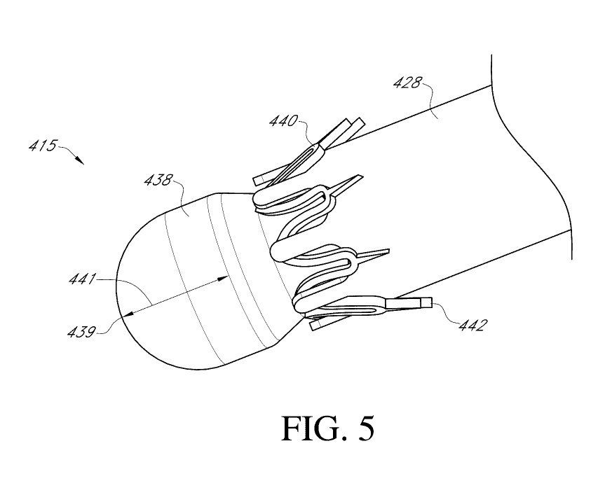

An existing embodiment of the device is shown in FIG. 5. FIG. 5 shows a device for diverticulum inversion with fixed atraumatic tip 415. The distal end of the device for diverticulum inversion with fixed atraumatic tip 415 includes a clip tube 428, a closure clip 440 with clip tines 442, and an atraumatic tip 438. The atraumatic tip 438 is connected to the distal end of the clip tube 428 and is distal to the closure clip 440. In this existing embodiment, the device for diverticulum inversion with fixed atraumatic tip 415 has a distal end 439 that is a fixed distance 441 from the clip tines 442 of closure clip 440. The atraumatic tip 438 is used to invert the diverticulum shown in FIGS. 1-3 above. In order to properly place the clip tines 442, the diverticulum must be fully inverted and the diverticulum tissue stretched away from the ostium so as to position the closure clip 440 to engage the diverticulum and ostial tissue simultaneously—enabling a complete closure and sealing off of the diverticulum. When a diverticulum presents an inverted depth deeper than the fixed distance 441, the existing embodiment of the device cannot manage the orientation of the tissue in order to engage the closure clip 440. As can be seen in the dimensional presentations of diverticulum 400 and dimensional presentations of inverted diverticulum 412, because of the variation of the shape and sizes of various diverticulum and ostium 414, there is a need for a device for treating diverticulum with a variable distance between the tip of the device used to invert the diverticulum and the location of the clip on the device prior to deploying the clip into the tissue surrounding the ostium 414.

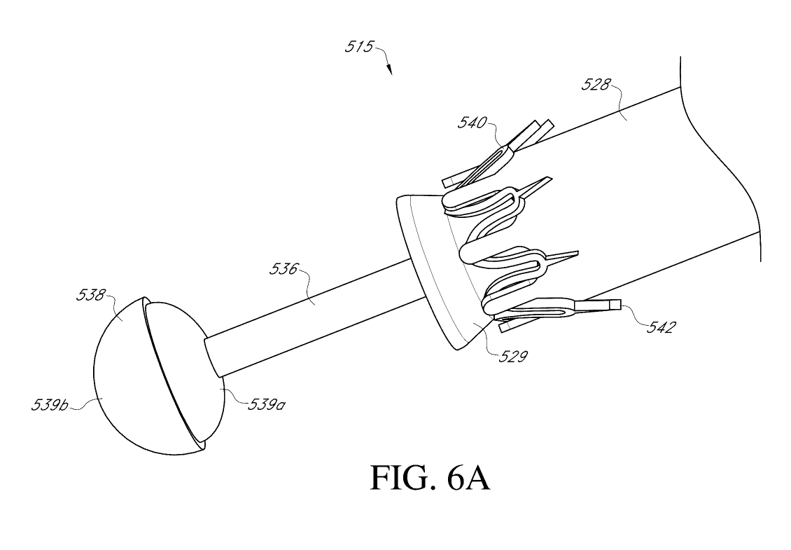

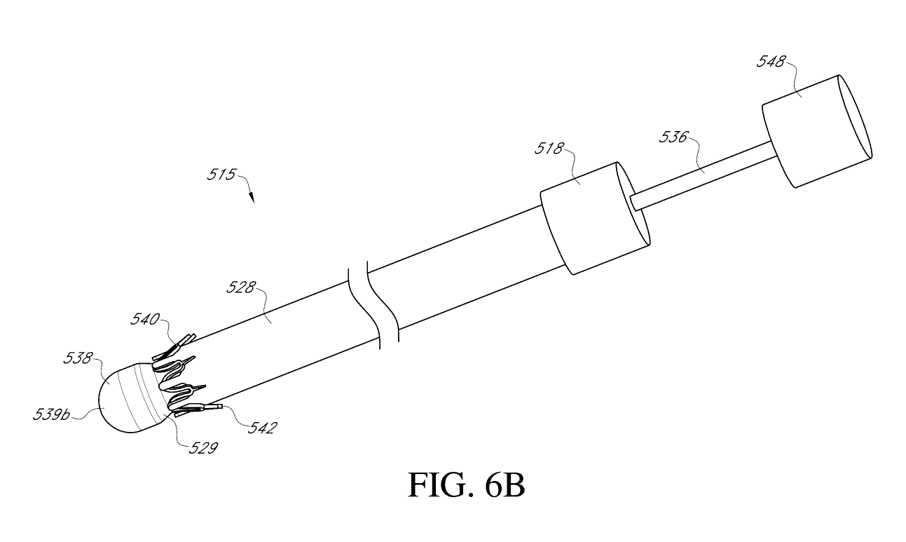

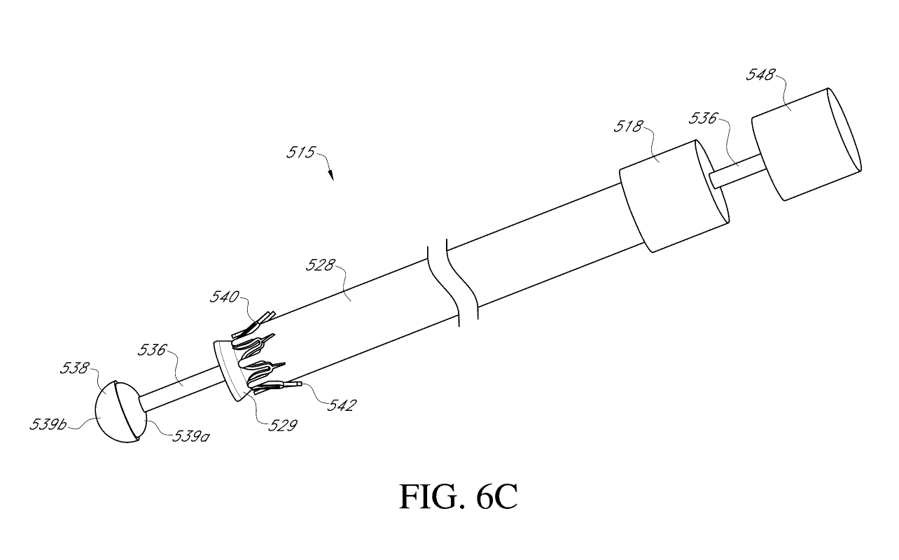

FIGS. 6A-C depicts a device for diverticulum inversion with extendable atraumatic tip 515. As seen in FIGS. 6A and B, the device for diverticulum inversion with extendable atraumatic tip 515 is designed to extend the inverted diverticulum tissue further away from the closure clip 540 in order to bring the ostium of the inverted diverticulum in proximity with the clip. In this embodiment, the device for diverticulum inversion with extendable atraumatic tip 515 includes a clip tube 528, a push rod 536, an atraumatic tip 538 and a user actuated structure 548. The clip tube 528 is disposed co-axially about the push rod 536 and has a closure clip 540 that is attached to the distal end of the clip tube 529 such that the clip tines 542 of the closure clip 415 extend proximally to the distal end of the clip tube 529. The push rod 536 has an atraumatic tip 538 attached to the distal end, a user actuated structure 548 attached to the proximal end, and extends proximally from the device proximal end 518. The push rod 536 is movably attached to the clip tube 528 such that it can move laterally relative to the clip tube 528. Prior to actuating the user actuated structure 548, the atraumatic tip 538 is located on the push rod 536 such that the proximal end 539a of the atraumatic tip 538 lies flush to the distal end of the clip tube 529.

FIG. 6C shows the device for diverticulum inversion with extendable atraumatic tip 515 in operation. In operation, a user can actuate the user actuated structure 548 to move the push rod 536 in a distal direction so as to advance the atraumatic tip 538. This increases the relative distance between the distal end 539b of the atraumatic tip 538 and the clip tines 542. In some embodiments, the user actuated structure 548 allows the user to have full control of the atraumatic tip 538 such that the user can determine the desired distance for inversion and control the relative position of the closure clip 540. In other embodiments, the atraumatic tip 538 is spring loaded in a manner such that the user actuated structure 548 is a button or trigger that can be depressed or activated respectively.

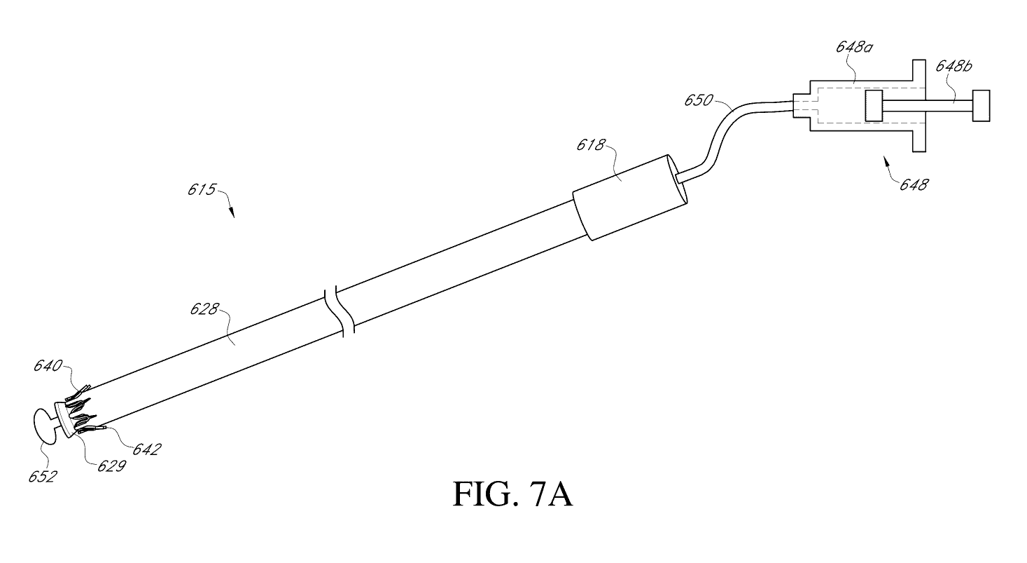

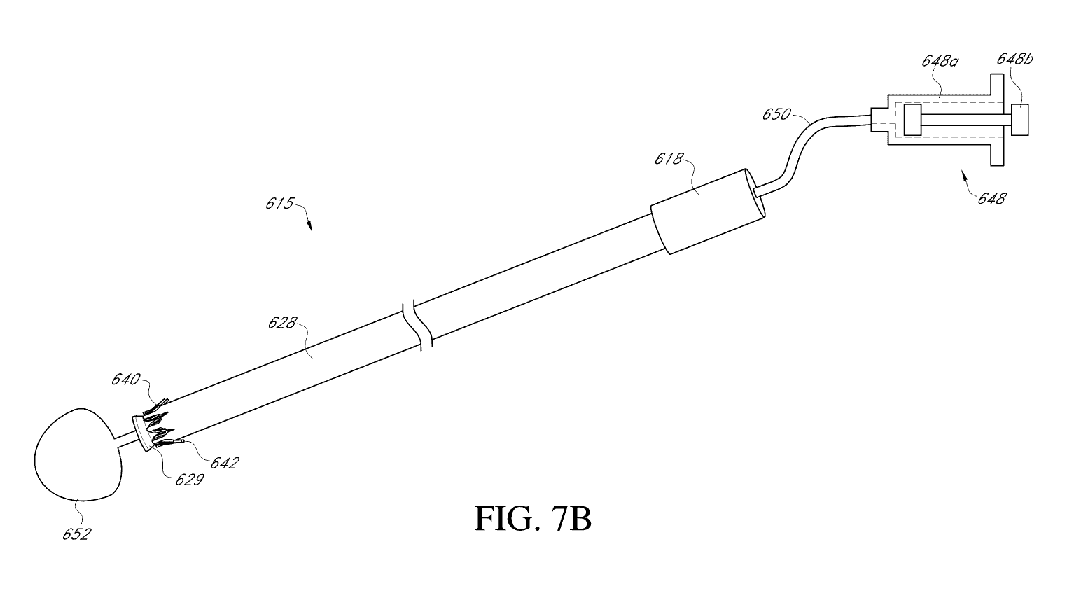

FIGS. 7A-B shows a device for diverticulum inversion with inflatable balloon tip 615, another embodiment of a device for treating diverticulum with a device tip that has a variable distance from the closure clip. The device for diverticulum inversion with inflatable balloon tip 615 has a distendable atraumatic tip. Distension of the tip effectively occupies the excess volume of the diverticulum, thereby bringing the closure clip 640 in favorable proximity to the ostium of the inverted diverticulum. This variation manages the depth of the inverted diverticulum by managing the volume of the space created by the inverted tissue. In this embodiment, the device for diverticulum inversion with inflatable balloon tip 615 includes a clip tube 628, a lumen 650, a balloon 652, and a user actuated structure 648. The clip tube 628 is disposed co-axially about the lumen 650 and has a closure clip 640 that is attached to the distal end of the clip tube 629 such that the clip tines 642 of the closure clip 640 are proximal to the distal end of the clip tube 629. The lumen 650 extends along the length of the clip tube 628 and is fluidly connected to a balloon 652 at the distal end and is fluidly connected to a user actuated structure 648 at the proximal end of the lumen 650. The lumen 650 can extend from the device proximal end 618 to connect with the user actuated structure 648. In some embodiments the user actuated structure 648 is a syringe 648a with a plunger 648b.

FIG. 7B shows the device for diverticulum inversion with inflatable balloon tip 615 in operation. In operation, the balloon 652 is inflated through the user actuated structure 648. In the embodiment shown here, the user actuated structure 648 is a syringe 648a with a plunger 648b, however other filling mechanisms and structures can be used as the user actuated structure 648. As shown in FIG. 7B, the user depresses the plunger 648b of the syringe 648a such that fluid contained within the syringe 648a is pushed through the lumen 650 and into the balloon 652. As the plunger 648b is incrementally depressed, the balloon 652 is inflated in a controlled manner. As the balloon 652 is inflated, the balloon 652 expands to fill the inverted diverticulum and brings the clip tines 642 of the closure clip 640 in proximity to the tissue of the ostium. This method of distention of the inverted diverticulum has the advantage of completely filling and controlling the inverted diverticulum from the inside using a method that allows for visualization of the tissue during and after the distention, prior to placement of the closure clip 640.

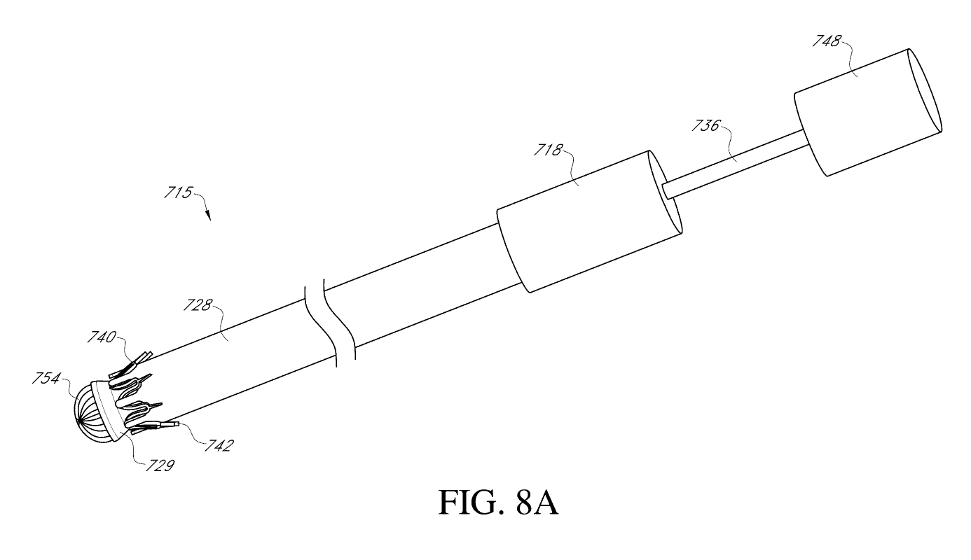

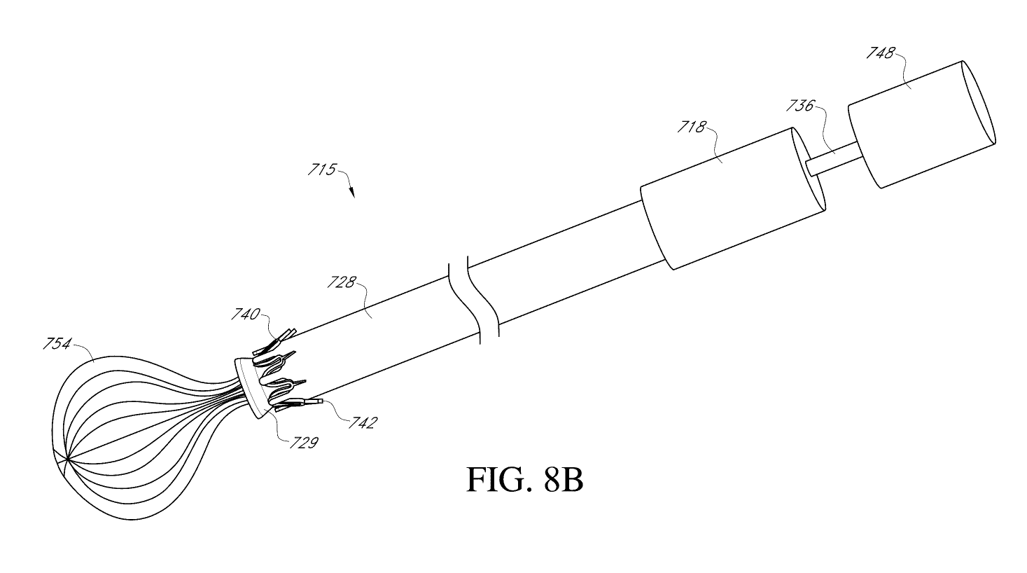

FIGS. 8A-B shows a device for diverticulum inversion with expandable tip 715 that is similar to the embodiment shown in FIGS. 7A-B. In this embodiment, the device for diverticulum inversion with expandable tip 715 has a distentable atraumatic tip that brings the closure clip 740 in favorable proximity to the ostium of the inverted diverticulum. However, the distension is accomplished by an expandable tip 754 constructed from a shape memory metal or polymer cage/mesh instead of a balloon 652. The device for diverticulum inversion with expandable tip 715 includes a clip tube 728, a push rod 736, an expandable tip 754, and a user actuated structure 748. The clip tube 728 is disposed coaxially about the push rod 736 and has a closure clip 740 that is attached to the distal end of the clip tube 729 such that the clip tines 742 of the closure clip 740 are proximal to the distal end of the clip tube 729. The push rod 736 has an expandable tip 754 attached to the distal end and a user actuated structure 748 attached to the proximal end. The push rod 736 is movably attached to the clip tube 728 such that it can move laterally relative to the clip tube 728 allowing the push rod 736 to protrude from the device proximal end 718. Prior to actuating the user actuated structure 748, the expandable tip 754 is retracted in the distal end of the clip tube 729 such that only the distal end of the expandable tip 754 protrudes.

FIG. 8B shows the device for diverticulum inversion with expandable tip 715 in operation. In operation, the expandable tip 754 is expanded by the user actuated structure 748. In the embodiment shown in FIG. 8B, the user actuated structure 748 is actuated by user by advancing the push rod 736 in a distal direction. This causes the expandable tip 754 to protrude from the distal end of the clip tube 729 distal end of the clip tube 729. As discussed above, in some embodiments, the expandable tip 754 is composed of a shape memory metal or polymer cage/mesh. In some embodiments, the expandable tip 754 is composed of a metal or polymer with elastic properties. In some embodiments, the expandable tip 754 can be composed of materials such as nitinol, cobalt-chromium, polyurethane, polyethylene terephthalate, or polyethyleneoxide. In other embodiments, the expandable tip 754 can be an expandable mesh, basket, or braided structure. In other embodiments, the expandable tip 754 can form a spheroid or a football shape. Therefore, as the expandable tip 754 is pushed outwards by the attached push rod 736, it returns to its expanded shape. As with the balloon 652 of the device for diverticulum inversion with inflatable balloon tip 615, this shape memory expandable tip 754 expands to fill the inverted diverticulum and brings the clip tines 742 of the closure clip 740 in proximity to the tissue of the ostium. This method of distention of the inverted diverticulum fills and controls the inverted diverticulum from the inside and allows the physician a better view of the tissue during and after the distention. This provides for better placement of the closure clip 740.

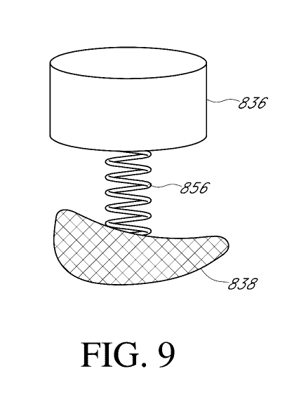

In some embodiments, as illustrated in FIG. 9, the inverted diverticulum can be expanded by a device distal end with an atraumatic tip 838 that can be attached to the push tube 836 by a spring 856. The extension of the atraumatic tip 838 from the push tube 836 can be done automatically as the distal end of the device is inserted. In other examples, the spring 856 can be activated to push the atraumatic tip 838 in a distal direction through a user actuated mechanism. The distance between the atraumatic tip 838 and the push tube 836 can be fixed or a set distance.

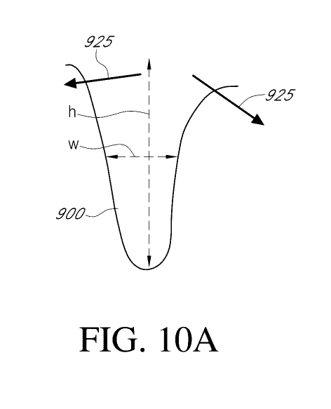

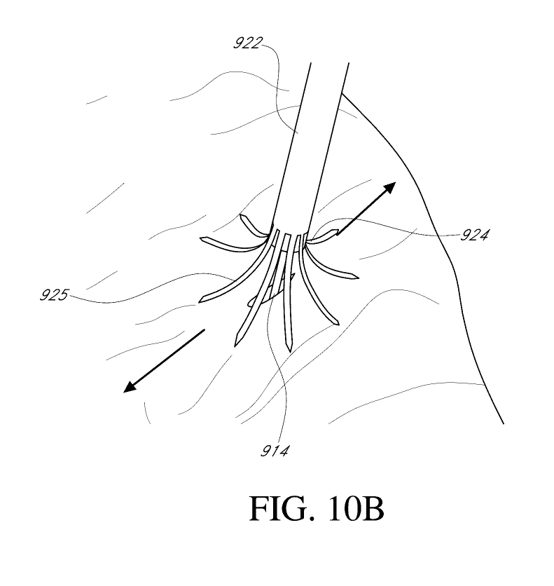

Another problem faced in the inversion of diverticulum is the oval-shape of the ostium of the inverted diverticulum that interferes with the effective treatment of the target diverticulum. As a result of the orientation of the underlying muscle fibers, once a diverticulum is inverted, the ostium will often transform from a round shape to an oval shape. FIGS. 10A-B provide an example of an inverted diverticulum with an inverted diverticulum with an oval-shaped ostium. As can be seen in FIG. 10A, the inverted diverticulum 900 has an inverted diverticulum diameter width w that gradually decreases along the inverted diverticulum height h. As a result of the uneven distribution of tissue, as can be seen in FIG. 10B, as the ostium 914 is inverted, the shape of the ostium 914 turns more oval in the direction and along the axis of the underlying muscle fibers in the muscularis layer of the colon. If the ostium 914 is oval and the basket shaft 922 with basket 924 is circular (forming a circle with the basket tines 925), the tissue surrounding the ostium 914 will not be gathered in a uniform fashion, making securing the tissue a challenge. As well, if the ostium 914 is oval in shape and the closure clip is round, the closure clip will not have uniform purchase on the tissue of the ostium 914 in order to secure tissue in the manner intended. This would therefore cause the treatment of the diverticulum to fail.

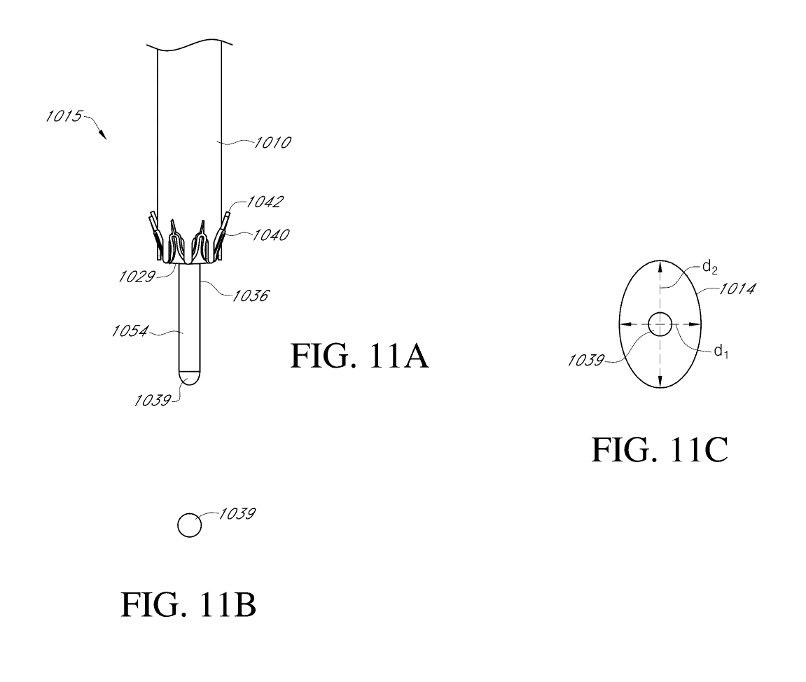

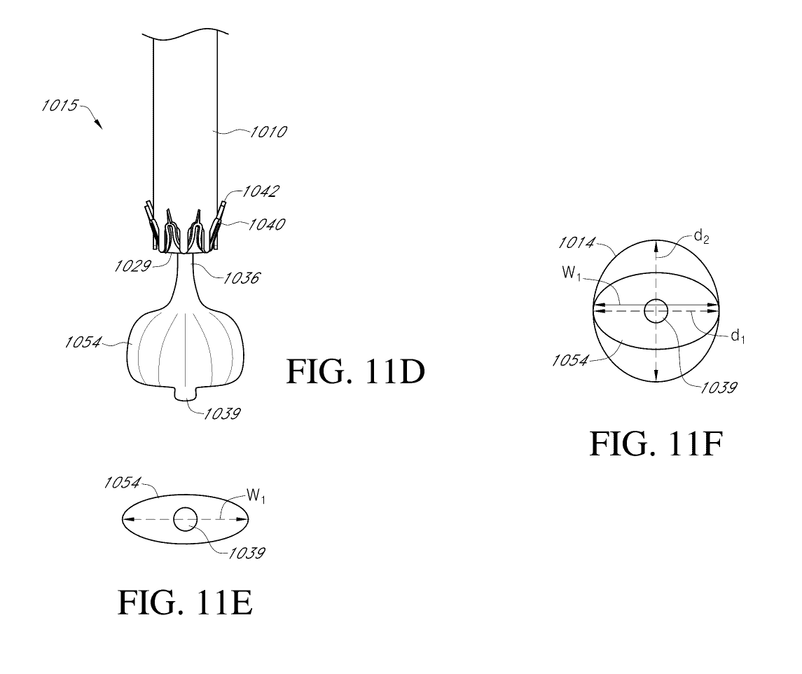

The devices described in FIGS. 11A-F and 12A-D provide embodiments of devices that include additional ellipsoid shapes to the distal end of the diverticulum inverting portion of the device. As will be discussed in more detail below, the tip of the device for diverticulum inversion has a blunt distal end used to invert the diverticulum. Once the diverticulum is inverted, the tip is deployed to form an ellipsoid shape inside the inverted diverticulum. The physician can then rotate the device to counteract the oval shape of the diverticulum with the ellipsoid shape of the distal end of the device. As a result of this repositioning, the ostium of the inverted diverticulum will become more round in shape. Such a conformation will allow the tissue gathering basket to be more evenly engaged around the diverticulum and the tissue more evenly recruited in preparation for clip placement. Following clip placement, the distal end of the device can be collapsed and withdrawn from the site of treatment.

FIGS. 11A-F show a device for diverticulum inversion with ellipsoid expandable tip 1015. This is similar to the structure and operation of the device for diverticulum inversion with expandable tip 715 shown in FIGS. 8A-B. The device for diverticulum inversion with ellipsoid expandable tip 1015 is seen with a clip tube 1010, push rod 1036, and expandable tip 1054. The clip tube 1010 is disposed about the push rod 1036 and includes a closure clip 1040 attached at the distal end of the clip tube 1029 such that the clip tines 1042 of the closure clip 1040 are pointed proximal to the distal end of the clip tube 1029. The push rod 1036 has an expandable tip 1054 attached to the distal end and a user actuated structure (not pictured) attached to the proximal end. The push rod 1036 is movably attached to the clip tube 1010 such that the push rod 1036 can cause the attached expandable tip 1054 to protrude and retract from the distal end of the clip tube 1029. Prior to actuating the user actuated structure described above, the expandable tip 1054 is retracted in the distal end of the clip tube 1029 such that only the distal end 1039 of the expandable tip 1054 protrudes. FIG. 11A shows a side view of the expandable tip 1054 before it is expanded and FIG. 11B shows an image of the distal end 1039 of the expandable tip 1054 before it is expanded. In operation, the distal end 1039 of the expandable tip 1054 is used to invert the diverticulum. FIG. 11C shows a top cross-sectional view of the inverted diverticulum with ostium 1014 after the distal end 1039 has inverted the diverticulum. As pictured in FIG. 11C, the dimensions of the ostium 1014 after inversion resemble that of an oval, with the inverted diverticulum diameter d2 longer than the inverted diverticulum diameter d1.

FIGS. 11D-F show the device for diverticulum inversion with ellipsoid expandable tip 1015 in operation. In operation, the expandable tip 1054 is expanded by the user actuated structure. In the embodiment shown in FIG. 11D, the user actuated structure advances the push rod 1036 in a distal direction. This causes the expandable tip 1054 to protrude from the distal end of the clip tube 1029. As discussed above, in some embodiments, the expandable tip 1054 is composed of a shape memory metal or polymer cage/mesh. In some embodiments, the expandable tip 1054 is composed of a metal or polymer with elastic properties. In some embodiments, the expandable tip 1054 can be composed of materials such as nitinol, cobalt-chromium, polyurethane, polyethylene terephthalate, or polyethyleneoxide. In other embodiments, the expandable tip 1054 can be an expandable mesh, basket, or braided structure. In other embodiments, the expandable tip 1054 can form a spheroid or a football shape. Therefore, as the expandable tip 1054 is pushed outwards by the push rod 1036, it returns to its expanded shape. As discussed above, the expandable tip 1054 expands to fill the inverted diverticulum and brings the clip tines 1042 of the closure clip 1040 in proximity to the tissue of the ostium 1014. Once the expandable tip 1054 has been expanded, it has an expandable tip width w1 that can be used to expand the inverted diverticulum diameter d1 of the ostium 1014. FIG. 11E shows an end view of the expandable tip 1054 once expanded. The expandable tip 1054 has an ellipsoid shape with an expandable tip width w1 along its longest side. FIG. 11E shows a top cross-sectional view of the expandable tip 1054 once it is expanded in the inverted diverticulum and the effect it has on the ostium 1014. In operation, once the expandable tip 1054 is expanded in the inverted diverticulum, the physician can rotate the expandable tip 1054 such that the expandable tip width w1 aligns to expand the inverted diverticulum diameter d1. This causes the ostium 1014 to become more circular which provides for better tissue gathering and clip placement.

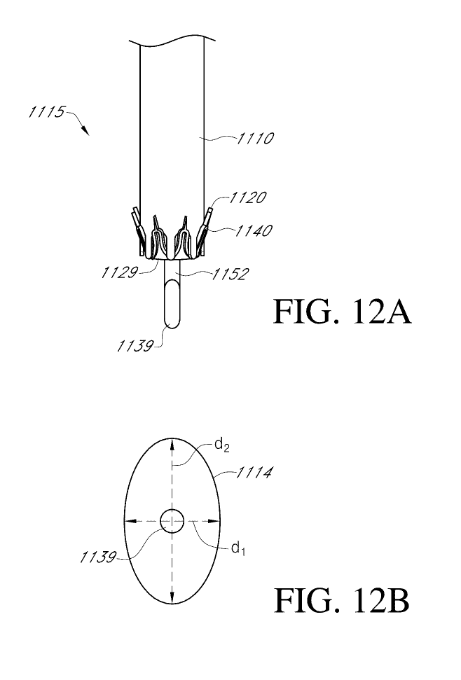

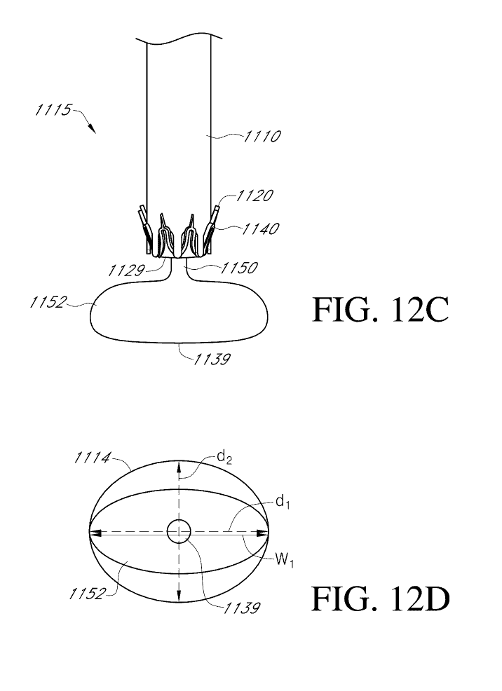

FIGS. 12A-D show a device for diverticulum inversion with ellipsoid inflatable tip 1115. The device for diverticulum inversion with ellipsoid inflatable tip 1115 serves the same function as the embodiment shown in FIGS. 11A-D except the distal end is a balloon 1152 instead of an expandable tip 1054. The device for diverticulum inversion with ellipsoid inflatable tip 1115 includes a clip tube 1110, a lumen 1150, a balloon 1152, and a user actuated structure (not pictured here). The clip tube 1110 is disposed co-axially about the lumen 1150 and has a closure clip 1140 that is attached to the distal end of the clip tube 1129 such that the clip tines 1120 of the closure clip 1140 are proximal to the distal end of the clip tube 1129. The lumen 1150 extends along the length of the clip tube 1110 and is fluidly connected to the balloon 1152 at the distal end and is fluidly connected to a user actuated structure at the proximal end. The user actuated structure can be any structure that provides for user controlled transfer of fluid to the balloon 1152. In some embodiments this is a syringe and plunger (as seen in FIGS. 7A-B). FIG. 12A shows a side view of the balloon 1152 before it is expanded. In operation, the distal end 1139 of the balloon 1152 is used to invert the diverticulum. FIG. 12B shows a top cross-sectional view of the inverted diverticulum with ostium 1114. Upon inversion, the ostium 1114 has dimensions that resemble an oval, with the inverted diverticulum diameter d2 longer than inverted diverticulum diameter d1.

FIGS. 12C-D shows the device for diverticulum inversion with ellipsoid inflatable tip 1115 in operation. In operation, the balloon 1152 is expanded by the user actuated structure. In one embodiment, the user actuated structure can be the syringe and plunger from FIGS. 7A-B where fluid is pushed into the lumen 1150 by the plunger. As the user actuated structure is used, fluid is pushed through the lumen 1150 into the balloon 1152. As discussed above, the balloon 1152 expands to fill the inverted diverticulum and brings the clip tines 1120 of the closure clip 1140 in proximity to the ostium 960. Once the balloon 1152 has been expanded, it has an expandable tip width w1 that can be used to expand the inverted diverticulum diameter d1 of ostium 1114. FIG. 9D shows a top cross-sectional view of the balloon 1152 once it is expanded into the inverted diverticulum and the effect it has on the ostium 1114. In operation, once the balloon 1152 is expanded in the inverted diverticulum, the physician can rotate the balloon 1152 such that the expandable tip width w1 aligns to expand the inverted diverticulum diameter d1. This causes the ostium 1114 to become more circular which provides for better tissue gathering and clip placement.

While the description generally refers to colonoscopes and treatments within a colon, the devices and methods described herein are not limited to applications within a colon. They can be used to invert and/or treat outpocketings (e.g., diverticula, aneurisms, etc.) in any body lumen. Any reference to a colonoscope should be understood to be applicable to endoscopes generally, and similarly, any reference to a colon should be understood to be applicable to any body lumen.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A device for inverting a diverticulum comprising:
a device body;
a pushing apparatus comprising a rod and a blunt end, wherein the rod is disposed coaxially within the device body and the blunt end is located at the distal end of the rod; and
a user actuated structure that provides control of the pushing apparatus.

2. The device of claim 1 wherein the pushing apparatus is movable along the length and relative to the device body, and wherein the user actuated structure translates the pushing apparatus in a distal direction such that the rod extends from the distal end of the device body.

3. The device of claim 1 wherein the pushing apparatus is spring loaded and the user actuated structure comprises a button or trigger configured to interact with the spring loaded pushing apparatus when actuated.

4. The device of claim 1 wherein the rod comprises a lumen and the blunt end comprises an expandable balloon, wherein the user actuated structure is a syringe configured to inject and withdraw fluid from the lumen to inflate or deflate the balloon.

5. The device of claim 4 wherein the user actuated structure is a button or trigger activated syringe

6. The device of claim 4 wherein the balloon is oval-shaped.

7. The device of claim 1 wherein the blunt end is a basket capable of expanding or collapsing as the pushing apparatus is extended or withdrawn from the distal end of the device body.

8. The device of claim 7 wherein the basket is composed of a metal with elastic properties such as nitinol or cobalt-chromium.

9. The device of claim 7 wherein the basket is composed of a polymer with elastic properties such as polyurethane, polyethylene terephthalate, or polyethyleneoxide.

10. The device of claim 7 wherein the basket is oval-shaped when expanded.

11. The device of claim 7 wherein the user actuated structure is a sliding member, button or a trigger.

12. A method for inverting a diverticulum comprising:
positioning a device along an outer wall of a colon at a diverticulum, wherein the device comprises a device body, a pushing apparatus, and a user actuated structure, wherein the device body is disposed about the pushing apparatus and the user actuated structure is configured to interact with the pushing apparatus;
inverting the outer surface of the diverticulum with the distal end of the device such that the distal end of the device is partially disposed within the inverted portion of the diverticulum; and
actuating the user actuated structure such that the pushing apparatus interacts with the diverticulum to further invert the diverticulum.

13. The method of claim 12 wherein the pushing apparatus comprises a rod and a blunt end attached to the distal end of the rod, wherein actuating the user actuated structure moves the pushing apparatus in a distal direction such that the blunt end extends from the distal end of the device body and further inverts the diverticulum.

14. The method of claim 13 wherein the pushing apparatus is spring loaded and the user actuated structure is a button or trigger configured to interact with the spring loaded pushing apparatus such that actuating the user actuated structure comprises depressing the button or activating the trigger.

15. The method of claim 14 further comprising retracting the pushing apparatus such that the rod and blunt end move in a proximal direction such that the rod withdraws into the device body.

16. The method of claim 12 wherein the pushing apparatus comprises a lumen coaxially disposed within the device body and an expandable balloon located at the distal end of the lumen, and the user actuated structure is a syringe configured to interact with the proximal end of the lumen; and wherein actuating the user actuated structure to further invert the diverticulum comprises depressing a plunger of the syringe such that fluid is injected into the lumen and inflates the expandable balloon.

17. The method of claim 16 further comprising retracting the plunger of the syringe such that fluid is pulled out of the lumen and deflates the expandable balloon.

18. The method of claim 17 wherein the expandable balloon is oval-shaped and the method further comprise the step of rotating the inflated expandable balloon such that the cross-section of the inverted diverticulum becomes increasingly circular.

19. The method of claim 12 further wherein the pushing apparatus comprises a rod and a basket attached to the distal end of the rod, wherein actuating the user actuated structure to further invert the diverticulum comprises moving the pushing apparatus in a distal direction such that the basket extends from the distal end of the device body and expands.

20. The method of claim 19 further comprising retracting the pushing apparatus such that the rod and the basket move in a proximal direction such that the basket collapses into the distal end of the device body.

21. The method of claim 20 wherein the basket is oval-shaped and the method further comprising the step of rotating the expanded basket such that the cross-section of the inverted diverticulum becomes increasingly circular.

22. The method of claim 19 wherein the user actuated structure is a sliding member, button, or a trigger.
